(19) United States
(12) Patent Application Publication
(10) Pub. No.: US 2024/0148723 A1
(43) Pub. Date: May 9, 2024

(54) MYCOBACTERIUM TUBERCULOSIS COMPOSITIONS AND METHODS

(71) Applicant: Wanliang Shi, Towson, MD (US)

(72) Inventor: Wanliang Shi, Towson, MD (US)

(21) Appl. No.: 18/548,804

(22) PCT Filed: Mar. 18, 2022
(86) PCT No.: PCT/US22/71222

§ 371 Date: Sep. 1, 2023

Related U.S. Application Data
(60) Provisional application No. 63/249,865, filed on Sep. 29, 2021.
(60) Provisional application No. 63/164,489, filed on Mar. 22, 2021.

(51) Int. Cl. A61K 31/4965; A61K 47/18; A61P 31/06; G01N 33/543; G01N 33/569

(52) U.S. Cl. CPC A61K 31/4965; A61K 47/183; A61P 31/06; G01N 33/54386; G01N 33/5695

(57) ABSTRACT

Novel nutrient compositions for facilitating Mycobacterium tuberculosis growth and achieving activity of PZA at neutral pH (e.g., 6.8) are described. These compositions utilize conventionally unfavored growth nutrients, including certain amino acids, cholesterol, lactate, fatty acids, or ammonium salts that are associated with Mycobacterium tuberculosis persistence in vivo. The present application describes methods for using such compositions to provide a reliable method for PZA susceptibility testing in which all tuberculosis drug susceptibility tests can be integrated into an efficient and economical system. The present application further describes screening and identification of PZA-like drugs in vitro for tuberculosis treatment.

This application claims priority to U.S. Provisional Patent Application Ser. No. 63/164,489, filed on Mar. 22, 2021, and U.S. Provisional Patent Application Ser. No. 63/249,865, filed on Sep. 29, 2021, the contents of which are expressly incorporated herein by reference herein.

### FIELD

The present invention relates to nutrient compositions for Mycobacterium tuberculosis growth and their use for antibiotic susceptibility testing and drug discovery.

### BACKGROUND

Pyrazinamide (PZA) is a critical component of first-line drug combination therapy for tuberculosis (TB), including both susceptible and multi-drug resistant tuberculosis (MDR-TB). Inclusion of PZA has shortened the previous 9-12 months chemotherapy regimen to 6 months. PZA has also become an essential part of MDR-TB treatment regimens that include novel compounds now clinically available, such as bedaquiline and others. PZA is reported to be inactive against organisms in the growth phase in conventional culture media at neutral pH and has a sterilizing effect due to its significant activity against non-replicating “persister” organisms or semi-dormant slowly replicating bacilli at acid pH conditions (pH 5.5), thereby killing bacilli that are not eliminated by other TB drugs, such as those found in acidic regions of acute inflammation.

It can take several days to identify and perform an antibiotic susceptibility test (AST) to identify M. tuberculosis in a culture and determine the antimicrobial susceptibility profile for that M. tuberculosis, especially one that is resistant to one or more antibiotics. An AST assay provides a “Minimum Inhibitory Concentration” or “MIC” value for each antimicrobial agent tested on a microorganism and can thus provide information on which antimicrobial agents may be effective against the microorganism.

There are several shortcomings associated with PZA susceptibility tests performed in acidic conditions: (1) 10˜30% clinical isolates won't grow in acidic condition; (2) false PZA resistance results often occur due to large inoculum size or instability under acidic condition. In other words, acidic conditions were neutralized due to growth of bacteria; (3) there is 99% inhibition with standard protocols for TB drug susceptibility testing, but 90% inhibition with PZA does not provide sufficient certainty with regard to a patient's response to PZA treatment; (4) acidic conditions require a 10-fold higher inoculum for PZA susceptibility tests than with other TB drug susceptibility tests; (5) to date, there is no PZA susceptibility test on solid media, which is problematic considering that the agar proportion method is the gold standard for TB drug AST. In view of the foregoing, there is a need for reliable and improved compositions and methods for performing AST assays. The present application addresses deficiencies in the art and provides improved compositions and methods for M. tuberculosis growth for diagnosis of TB, antibiotic susceptibility testing, and screening new compounds for activity against M. tuberculosis.

### SUMMARY

The present application provides improved compositions and methods for TB diagnosis, culture-based AST of M. tuberculosis, and screening of drug compounds having anti-tuberculosis activity. More particularly, the present application describes compositions and conditions for achieving activity of PZA and other antibiotics at neutral pH (e.g., 6.8) in media using conventionally unfavored growth nutrients, and their use for antibiotic susceptibility tests (ASTs) and drug screening of compounds having anti-tuberculosis activity. Advantageously, the composition and methods described herein avoid problems with growth inhibition and false positives for resistance, which are observed in ASTs that are conventionally performed under acidic conditions. The compositions and methods described herein provide a reliable method for PZA susceptibility testing in which all TB drug susceptibility testing can be integrated into an efficient and economical system.

In one aspect, a nutrient composition for facilitating growth of M. tuberculosis comprises: (i) one or more nitrogen sources comprising one or more amino acids or salts thereof, alone or in combination with one or more ammonium salts; (ii) one or more carbon sources comprising glycerol, butyric acid, lactate, cholesterol, pyruvic acid, dextrose, citric acid, or a combination thereof; (iii) one or more salts comprising MgSO₄, KH₂PO₄, K₂HPO₄, NaH₂PO₄, or a combination thereof; and (iv) one or more supplements comprising ZnSO₄, pyridoxine hydrochloride, biotin, Tween 80, oleic acid, albumin, ferric ammonium citrate, catalase, or a combination thereof; wherein the amino acids comprise low concentration of asparagine, aspartic acid, glutamine or glutamic acid, and wherein the composition is formulated to support antibiotic susceptibility testing of M. tuberculosis at a pH between 6.2 to 8.0 at 35-37° C.

In another aspect, the present application provides a method for determining drug susceptibility of an antibiotic for treating a M. tuberculosis infection, comprising the steps of: (a) inoculating a first medium comprising a nutrient composition of the present application (as described above) with a clinical sample of M. tuberculosis; (b) incubating the inoculated first medium in the presence or absence of the antibiotic under neutral pH conditions for a period of time and temperature sufficient to observe growth of the test sample in the absence of the antibiotic; (c) comparing the growth of the test sample in the presence or absence of the antibiotic; and (d) determining whether the test sample is sensitive or resistant to the antibiotic based on the comparison in step (c).

In another aspect, the present application provides a method for screening a plurality of test compounds or drugs for their ability to inhibit the growth of M. tuberculosis. In one embodiment, the method comprises the steps of: (a) inoculating a culture medium comprising a nutrient composition described in the present application with a M. tuberculosis isolate to form a control culture; (b) inoculating in parallel a plurality of secondary cultures comprising the M. tuberculosis in step (a) to from a plurality of test cultures, wherein each of the plurality of test cultures comprises a different test compound in the medium comprising the composition in step (a); (c) incubating in parallel the first culture and the test cultures under neutral pH conditions for a period of time and temperature sufficient to observe growth of the control sample in the absence of the different test compounds in step (b); (d) comparing the growth of the M. tuberculosis isolate in the presence or absence of the test compound; and (e) determining whether one or more of the test compounds inhibit growth of the M. tuberculosis isolate, based on the comparison in step (d). The drug screening method of the present application may be carried out using any one of a number of nutrient compositions and/or culture conditions described herein.

In another aspect, the present application provides a pharmaceutical composition for treatment or prevention of tuberculosis comprising a compound set forth in Table 12 in combination with a pharmaceutically acceptable carrier and other TB drugs.

In another aspect, the present application provides a method for treatment of M. tuberculosis, comprising administering to a subject in need thereof a compound set forth in Table 12 in combination with a pharmaceutically acceptable carrier.

These and other aspects of the present invention are described in more detail in the description of the invention set forth below.

### BRIEF DESCRIPTION OF THE DRAWINGS

FIG. 1 shows in vitro growth of M. tuberculosis H37Ra at neutral pH 6.8 and its susceptibility to PZA when cholesterol was used as the primary carbon source at pH 6.8 in a defined medium containing 0.25 g/L ammonium chloride, 0.5 g/L KH₂PO₄, 0.5 g/L MgSO₄, 30 mg/L ferric ammonium citrate, 0.5 mg/L biotin, 1 mg/L pyridoxine hydrochloride, 0.5 mg/L ZnSO₄. The cholesterol was present in the medium at various concentrations, including 160 mg/L (A), 80 mg/L (B) and 40 mg/L (C). The MIC of PZA in these conditions was determined following a 20-day incubation at 37° C. via the broth microdilution method utilizing a 96 well plate. The MIC was identified as the lowest growth inhibition of PZA concentration in the well and is labeled with a box encircling the wells corresponding to that PZA concentration.

FIG. 2 shows in vitro growth of M. tuberculosis H37Ra at neutral pH 6.8 and its susceptibility to PZA when one of 20 kinds of amino acid was used as the primary nitrogen source in a defined medium containing 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄. The MIC of PZA was determined at 37° C. following a 20-day incubation at 37° C. via the broth microdilution method utilizing a 96 well plate. The MIC with the lowest growth inhibition of PZA concentration in the well and is labeled with a box encircling the wells corresponding to that PZA concentration.

FIG. 3, panel A shows growth inhibition of M. tuberculosis H37Ra by PZA at pH 6.8 on an agar plate containing 2.0 g/L, L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.5 g/L albumin, 0.2 g/L dextrose, 0.0003 g/L catalase (ADC) and 1.5% agar (panel A); on an agar plate containing 0.25 g/L, ammonium chloride, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.5 g/L albumin, 0.2 g/L dextrose, 0.0003 g/L catalase and 1.5% agar (panel B); on an agar plate containing 0.25 g/L, ammonium acetate, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.5 g/L albumin, 0.2 g/L dextrose, 0.0003 g/L catalase and 1.5% agar (panel C); and on an Middlebrook 7H11 agar plate supplemented with 5.0 g/L albumin, 2.0 g/L dextrose, 0.003 g/L catalase (panel D). 6 mm Paper disc I, DMSO control; 6 mm Paper disc II containing 400 μg PZA; 6 mm Paper disc III containing 800 μg PZA; 6 mm Paper disc IIII containing 1600 μg PZA.

FIG. 4 shows growth inhibition by PZA at pH 6.8 on agar plates containing 2.0 g/L, L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.5 g/L albumin, 0.2 g/L dextrose, 0.0003 g/L catalase and 1.5% agar seeded with M. tuberculosis H37Ra (panel A); M. tuberculosis PZA-resistant strain #6 strain (PncA mutation L159P) (panel B); M. tuberculosis PZA-resistant strain #78 strain (PncA mutation C138R) (panel C); M. tuberculosis PZA-resistant strain #98 strain (PncA mutation W68R) (panel D); M. tuberculosis PZA-resistant strain #106 strain (PncA mutation D49G) (panel E); and M. tuberculosis PZA-resistant strain #186 strain (PncA mutation G132A) (panel F). Paper disc I, 6 mm, DMSO control; Paper disc II, 6 mm paper disc containing 400 μg PZA; Paper disc III, 6 mm paper disc containing 800 μg PZA; Paper disc IIII, 6 mm paper disc containing 1600 μg PZA.

FIG. 5 shows pH changes by color following M. tuberculosis growth in a defined medium containing 2.0 g/L, L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, and 15 μg/ml phenol red. Row A, Unseeded control; Row B, M. tuberculosis H37Ra grown at 37° C. for 2 weeks.

FIG. 6 shows a determination of MICs for first-line TB drugs against M. tuberculosis H37Ra using the broth microdilution method using a defined medium at pH 6.8 containing 2.0 g/L, L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, at 37° C. for 2-week incubation. As shown in panel A, Two-fold serial dilutions of the drugs were added to the wells as shown in panel A (EMB, ethambutol; RIF, rifampin; PZA, pyrazinamide; INH, isoniazid). Wells in panel B did not include phenol red in the media; wells in panel C included 15 μg/ml of phenol red in the media.

FIG. 7 shows color changes when M. tuberculosis grow at pH 6.8 in defined media using broth dilution method for 2 weeks incubation at 37° C. Panel A shows a color change from light red to pink in media containing 2.0 g/L L-Ala, 0.25 g/L ammonium acetate, 0.2 g/L L-Leu, 0.2 g/L Met, 0.2 g/L L-Phe, 0.2 g/L L-Pro, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.0125% Tween 80 and 20 μg/ml phenol red. Panel B shows a color change from light red to brown in media containing 2.0 g/L L-Ala, 0.2 g/L L-Leu, 0.2 g/L Met, 0.2 g/L L-Phe, 0.2 g/L L-Pro, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.0125% Tween 80, and 20 μg/ml phenol red. Panel C shows no significant color change in media containing 2.0 g/L L-Ala, 0.02 g/L L-Gln, 0.08 g/L L-Leu, 0.08 g/L L-Phe, 0.08 g/L L-Ser, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.0125% Tween 80 and 20 μg/ml phenol red.

FIG. 8 shows MIC determinations of PZA against M. tuberculosis and color changes in different medium in the presence of phenol red by the broth microdilution method. Samples with the lowest PZA concentration exhibiting no visible growth are labeled with a red rectangle.

FIG. 9 shows a determination of MIC of PZA using the broth macrodilution susceptibility testing method for M. tuberculosis H37Ra (PZA^{S}) and PZA-resistant strain (PZA^{R}) in defined medium at pH 6.8 containing 2.0 g/L L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄.

### DETAILED DESCRIPTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only, and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated by reference in their entirety.

As used herein, the phrase “neutral conditions” is used with reference to pH conditions, specifically a pH range between 6.2 to 7.6. The phrase “acidic conditions” is used with reference to pH conditions, specifically a pH range less than 6.2. The phrase “basic conditions” is used with reference to pH conditions, specifically a pH range greater than 7.6.

As used herein, the phrase “primary nitrogen source” is used with reference to a principal nitrogen source in PZA-S1-Minimal or PZA-S1-Plus media having a media concentration of at least 0.01 g/L. Accordingly, this phrase does exclude the use of minor amounts of other nitrogen or nitrogen salt sources below this concentration.

As used herein, the phrase “primary carbon source” is used with reference to a principal carbon source in PZA-S1-Minimal or PZA-S1-Plus media having a media concentration of at least 0.5 g/L. Accordingly, this phrase does exclude the use of minor amounts of other carbon sources below this concentration.

As used herein, the term “PZA-S1-Minimal” is used with reference to a growth medium composition in liquid broth or agar plates containing 0.5 g/L KH₂PO₄, 0.5 g/L MgSO₄, 30 mg/L ferric ammonium citrate, 0.5 mg/L biotin, 1 mg/L pyridoxine hydrochloride, and 0.5 mg/L ZnSO₄. Unless otherwise noted, a “PZA-S1-Minimal” medium is supplemented with one or more amino acids and/or ammonium salts and at least one or more carbon sources selected from the group consisting of glycerol, citric acid, dextrose, and/or lipids, such as cholesterol, butyric acid, pyruvic acid and/or lactic acid; and optionally one or more supplements, such as ADC, Tween 80, and others described herein.

The term “PZA-S1-Plus” is used with reference to a PZA-S1-Minimal medium further supplemented as described above. 0.05% bovine albumin 0.02% dextrose, and 0.3 mg/L catalase (ADC) and 0.0125% Tween 80. As used herein, the term “PZA-S1-Plus” is used with reference to a PZA-S1-Minimal medium composition in liquid broth or agar plates which is to a further supplemented as described above such that it additionally includes e.g., one or more amino acids (e.g., 2 g/L L-Alanine alone or in combination with other amino acids); one or more carbon sources (e.g., 10 ml/L glycerol and others described herein; agar (e.g., 1.5% Bacto agar when applied to agar plates; and optionally one or more supplements, such as e.g., ADC (e.g., 0.02% dextrose and 0.05% bovine albumin and 0.3 mg/L catalase), Tween 80, and others described herein.

In one aspect, a nutrient composition for facilitating growth of M. tuberculosis, comprises: (i) one or more nitrogen sources comprising one or more amino acids or salts thereof, alone or in combination with one or more ammonium salts; (ii) one or more carbon sources comprising glycerol, butyric acid, lactate, cholesterol, pyruvic acid, dextrose, citric acid, or a combination thereof; (iii) one or more salts comprising MgSO₄, KH₂PO₄, K₂HIPO₄, NaH₂PO₄, or a combination thereof, and (iv) one or more supplements comprising ZnSO₄, pyridoxine hydrochloride, biotin, Tween 80, oleic acid, albumin, ferric ammonium citrate, catalase, or a combination thereof; wherein the amino acids comprise lower concentration of (comparing to conventional culture media) asparagine, aspartic acid, glutamine, or glutamic acid, and wherein the composition is formulated to support antibiotic susceptibility testing of M. tuberculosis at a pH between 6.2 to 8.0 at 35-37° C.

The nitrogen source(s) for use in the present application include one or more amino acids. In some embodiments, the one or more amino acids include Ala, Arg, Lys, Ser, Thr, Ile, Gly, His, Pro, Val, Leu, Met, Trp, Tyr, Phe or a combination thereof. In preferred embodiments, the amino acid is alanine. In some embodiments, asparagine, aspartic acid, glutamine and glutamic acid may be included at low concentrations below the concentrations used in conventional M. tuberculosis culture media.

In some embodiments, the nutrient composition is provided in a dehydrated or powdered form to provide specific concentrations for each component when dissolved in a suitable diluent, such as sterile water or PBS so that the dehydrated or powdered form as whole is present in the diluent at a 0.1%, 0.2%, 0.5%, 1%, 2%, 5%, or 10% concentration (w/v). Accordingly, in any of the compositions, where a media component is defined by a concentration or range pertaining to a working concentration, it should be understood, that the compositions of the present application also include compositions in which each component is present in a more concentrated amount so that upon dilution by a factor of e.g., 5-fold, 10-fold, 50-fold, 100-fold, 500-fold, 1000-fold, 5,000-fold, the working concentration of each component is achieved.

In view of the foregoing, in certain embodiments, the nutrient composition includes one or more amino acids formulated to provide a working concentration, wherein each amino acid is present in the diluent to provide a final concentration of at least, at least 0.2 g/L, 0.5 g/L, at least 1 g/L, at least 2 g/L, at least 3 g/L, at least 4 g/L, or at least 5 g/L. In other embodiments, one or more amino acids are formulated to provide a working concentration, wherein each amino acid is present in an amount less than 0.2 g/L, less than 0.5 g/L, less than 1 g/L, less than 2 g/L, less than 3 g/L, less than 4 g/L, or less than 5 g/L. In other embodiments, one or more amino acids are formulated to provide a working concentration, wherein each amino acid is present in an amount between 0.2 g/L and 4 g/L, between 0.5 g/L and 4 g/L, between 0.75 g/L and 3 g/L, between 1 g/L and 2 g/L, 1 g/L, 2 g/L, 3 g/L, 4 g/L, 5 g/L or any range thereof.

In some embodiments the nitrogen sources include one or more ammonium salts. Exemplary ammonium salts are selected from the group consisting of ammonium acetate, ammonium succinate, ammonium chloride, ammonium citrate dibasic, ammonium tartrate dibasic, ammonium formate, ammonium nitrate, and combinations thereof. In certain embodiments, the composition is formulated to provide one or more ammonium salts are formulated to provide a working concentration, wherein each ammonium salt is present in the diluent to provide a final concentration of at least 0.05 g/L, at least 0.1 g/L, at least 0.2 g/L, at least 0.4 g/L, or at least 1 g/L. In other embodiments, one or more ammonium salts are formulated to provide a working concentration, wherein each ammonium salt is present in an amount less than 0.1 g/L, less than 0.2 g/L, less than 0.5 g/L, less than 1 g/L, less than 2 g/L, less than 3 g/L, less than 4 g/L, or less than 5 g/L. In other embodiments, one or more ammonium salts are formulated to provide a working concentration, wherein each ammonium salt is present in an amount between 0.05 g/L and 1 g/L, between 0.1 g/L and 0.6 g/L, between 0.2 g/L and 0.4 g/L, 0.1 g/L, 0.15 g/L, 0.2 g/L, 0.25 g/L, 0.4 g/L, 0.5 g/L, 1 g/L, 2 g/L, 3 g/L, 4 g/L, 5 g/L or any range thereof.

The nutrient composition for facilitating growth of M. tuberculosis growth includes one or more carbon sources. Exemplary carbon sources include glycerol, citric acid cholesterol, butyric acid, lactic acid, dextrose, or a combination thereof. In some embodiments, the carbon source is a lipid. In some embodiments, the carbon source is a fatty acid, such as a C₁-C₈ fatty acid. In certain preferred embodiments, the one or more carbon sources include glycerol. In other preferred embodiments, the one or more carbon sources include cholesterol. In certain embodiments, one or more carbon sources are each formulated to provide a working concentration, wherein each carbon source is present in an amount of at least 0.1 g/L, at least 0.2 g/L, at least 0.5 g/L, at least 1 g/L, at least 2 g/L, at least 3 g/L, at least 4 g/L or at least 5 g/L. In other embodiments, one or more carbon sources are each formulated to provide a working concentration, wherein each carbon source is present in amount less than 0.1 g/L, less than 0.2 g/L, less than 0.5 g/L, less than 1 g/L, less than 2 g/L, less than 3 g/L, less than 4 g/L, or less than 5 g/L. In other embodiments, one or more carbon sources are each formulated to provide a working concentration, wherein each carbon source is present in an amount between 0.1 g/L and 5 g/L, between 0.5 g/L and 2.5 g/L, between 1 g/L and 2 g/L, 0.5 g/L, 1 g/L, 1.5 g/L, 2 g/L, 3 g/L, 4 g/L, 5 g/L or any range thereof.

In another aspect, the present application provides a medium comprising the nutrient composition for facilitating M. tuberculosis growth as described herein. In some embodiments, the medium is in a liquid form. In other embodiments, the medium is in a solid form. It should be understood that the term “medium,” as described herein, may be used to describe a culture medium, as in a broth or as contained in solidified agar. In some embodiments, the medium is described with reference to a solid, such as a solidified agar composition, a plate (containing e.g., an agar composition), a microtiter plate, a tube, a container which can contain a liquid medium etc.

In preferred embodiments, the medium comprises any one of the nutrient compositions described in the present application for facilitating M. tuberculosis growth, wherein the medium is a liquid or solid, and wherein the nutrient composition in liquid form or in solid form (e.g., in agar) has a pH between 6.2 to 8.0. In some embodiments, the pH is between 6.5 and 7.5, between 6.8 and 8.0, between 6.8 and 7.5, between 6.8 and 7.2, between 6.9 and 7.1, about 6.8, about 6.9, about 7.0, about 7.2, about 7.4 or any range thereof. The nutrient composition can include one or more buffers to provide any of the foregoing pH values or the nutrient composition can be formulated so that a sufficient amount of a suitable acid or base is added to achieve the desired pH.

The nutrient composition includes one or more supplementary nutrients. Exemplary supplementary nutrients include, for example, albumin, dextrose, catalase, ZnSO₄, pyridoxine hydrochloride, biotin, Tween 80, oleic acid, ferric ammonium citrate, and combinations thereof.

In some embodiments, to avoid microbial contamination, the nutrient composition includes one or more antimicrobial agents. Exemplary antimicrobial agents include cycloheximide, carbenicillin, polymyxin B, and trimethoprim.

In some embodiments, the nutrient composition includes a pH indicator, such as phenol red at a concentration of e.g., 5-20 μg/ml. Other pH indicators may be used so long as they change color in culture media at a pH range between 5.5 and 9.0. Additional pH indicators for use according to the present application include, but are not limited to neutral red, bromothymol blue, para-nitrophenol, cyanin, cresol red, and thymol blue.

In another aspect, the present application provides a method for determining drug susceptibility of an antibiotic for treating a M. tuberculosis infection, comprising the steps of: (a) inoculating a first medium comprising a nutrient composition of the present application (as described above) with a clinical test sample of M. tuberculosis; (b) incubating the inoculated first medium in the presence or absence of the antibiotic under neutral pH conditions for a period of time and temperature sufficient to observe growth of the test sample in the absence of the antibiotic; (c) comparing the growth of the test sample in the presence or absence of the antibiotic; and (d) determining whether the test sample is sensitive or resistant to the antibiotic based on the comparison in step (c).

In some embodiments, the method additionally includes the steps of: (a2) inoculating a second medium with the nutrient composition in step (a) with a M. tuberculosis control sample that is sensitive to the antibiotic; (b2) incubating the inoculated second medium in the presence or absence of the antibiotic under neutral pH conditions for a period of time and temperature sufficient to observe growth of control sample in the absence of the antibiotic; (c2) comparing the growth of the control sample with the growth of the test sample in media containing the antibiotic, and (d2) determining whether the test sample is sensitive or resistant to the antibiotic based on the comparison in step (c2), wherein a substantial growth inhibition of the control sample, and a substantial absence of growth inhibition of the test sample indicates that the test sample is resistant to the antibiotic, and wherein substantial growth inhibition of both the control sample and the test sample indicates that the test sample is sensitive to PZA.

In one embodiment, the comparing step is based on measuring the growth of the M. tuberculosis on an agar medium as compared to growth of the M. tuberculosis on the agar medium in the absence of the antibiotic. Techniques for measuring the growth of M. tuberculosis on the agar medium of the present invention include, but are not limited to, counting colonies on the plate under a microscope (e.g., a dissecting microscope). Typically, the number of colonies in a drug-containing plate segment or well is divided by the number of colonies grown on the drug-free control segment or well and multiplied by 100. If the percentage is greater than, or equal to, a pre-established drug-resistance level for the particular drug, then the culture is considered to be resistant to that drug. If the percentage is less than the pre-established drug-resistance level, then the culture is considered to be susceptible to that drug.

Determining susceptibility of a clinical M. tuberculosis isolate to an antibiotic is preferably based on susceptibility to a first-line antibiotic used against M. tuberculosis. Exemplary antibiotics for susceptibility testing include pyrazinamide, rifampin, rifapentine, isoniazid, ethambutol, bedaquiline, linezolid, moxifloxacin, and fluoroquinolone, delamanid, pretoinanid, amikacin, clofazimine, cycloserine, rifabutin, levofloxacin, ethionamide, p-aminosalicylic acid, streptomycin, and combinations thereof. However, the susceptibility testing may be applicable to any antibiotic known to exhibit antimicrobial properties against M. tuberculosis.

In some embodiments, the test sample is deemed be susceptible to the antibiotic where growth of the test sample in the presence of the antibiotic is less than a “pre-established drug-resistance level” for the antibiotic in the absence of the antibiotic. As used herein, the phrase “pre-established drug-resistance level” refers to a level of growth of a culture which represents the “break point” between a culture being resistant to the drug or susceptible to the drug. As described above, a critical concentration of a given drug can be determined for the drug in the agar medium of the present invention. The drug-resistance level of growth may be established by determining a percentage of growth on the critical concentration of the drug, as compared to the growth in the absence of the drug (i.e., 100%), below which the culture is considered to be susceptible to the drug. The drug-resistance level, or breakpoint, for tuberculosis drugs in an agar medium can readily be determined by those of skill in the art.

The AST and drug screening experiments described herein may employ a variety of different antibiotic concentrations. In some embodiments, the antibiotic is present at a concentration between 0.001 μg/ml and 1 mg/ml. In some embodiments, the antibiotic is present at a concentration between 0.001 μg/ml and 25 μg/ml, between 0.005 μg/ml and 20 μg/ml, between 0.005 μg/ml and 10 μg/ml, between 0.005 μg/ml and 5 μg/ml, between 0.01 μg/ml and 25 μg/ml, between 0.1 μg/ml and 10 μg/ml, or any of the foregoing integer values or range defined by the foregoing integer values. In some embodiments, the antibiotic (such as PZA) is present and a concentration between 0.2 μg/ml and 1 mg/ml, between 1 and 500, between 5 and 500, between 10 and 500, between 20 and 500, between 50 and 500, between 100 and 500, between 250 and 500, between 10 and 200, between 20 and 200, between 30 and 200, between 40 and 200, between 50 and 200, between 20 and 100, between 35 and 100, between 50 and 100 μg/ml, or any of the foregoing integer values or range defined by the foregoing integer values.

Generally, for the tuberculosis drugs disclosed herein, with the exception of PZA, the pre-established drug-resistance is the proportion of bacteria no response to antibiotic at given concentration exceeding 1% (these are international standards). Therefore, for testing on these drugs, if the growth of the M. tuberculosis on the agar medium containing the drug is less than 1% of the growth of the M. tuberculosis on the agar medium in the absence of the drug, the sample of M. tuberculosis is susceptible to the drug. The breakpoint for PZA is 10%, which constitutes a current international standard in liquid media.

Presently, there is no accepted PZA susceptibility test for solid media. For PZA susceptibility testing according to the present application, if the growth of the M. tuberculosis on the agar medium containing the PZA is less than 1% of the growth of the M. tuberculosis on the agar medium in the absence of PZA, then the M. tuberculosis is deemed to be susceptible to PZA. This test can be performed in liquid and solid agar with standard protocol parameters e.g., <1% growth in/on drug-containing media as compared to media without drug as being susceptible; or >1% growth in/on drug-containing media as compared to media without drug as being resistant.

Susceptibility testing according to the present application is carried out under neutral pH conditions. In some embodiments, the nutrient composition or culture medium has a pH between 6.2 to 8.0. In some embodiments, the pH is between 6.2 and 7.5, between 6.8 and 8.0, between 6.8 and 7.5, between 6.8 and 7.2, between 6.9 and 7.1, about 6.8, about 6.9, about 7.0, about 7.2, about 7.4 or any range thereof.

Generally, the M. tuberculosis cultures are grown under conditions (e.g., incubation time and temperature) sufficient for growth of M. tuberculosis, such as 37° C. for 1 to 6 weeks, 1 to 4 weeks, 2 to 4, weeks, 1 week, 2 weeks, 3 weeks, 4 weeks, 5 weeks, or 6 weeks.

The step of incubating typically occurs at a temperature of from about 35-37° C., typically in the dark, and can be performed in the absence of supplemental CO₂ (i.e., in normal air conditions). In one embodiment, the step of incubating occurs for at least 2 weeks, and preferably about 3 weeks, and is typically from about 3 weeks to about 6 weeks. An incubation of longer than 6 weeks is typically undesirable, since after 6 weeks, because this may preclude an accurate interpretation of the drug susceptibility test results.

In some embodiments, the susceptibility test is carried out in liquid media. In other embodiments, the susceptibility test is carried out using agar plates containing the nutrient composition of the present application. In some embodiments, a culture is initially cultured in a liquid medium, followed by plating onto an agar medium, which may result in shorter incubation times. Of course, the incubation period can be adjusted and monitored readily by those of skill in the art.

As noted above, the M. tuberculosis cultures can be grown as a liquid culture in tubes or microtiter plate wells, or on agar plates and the like. Additionally, the media can be modified to include any one of a variety of nitrogen sources, carbon sources, supplementary nutrients, antimicrobial agents, and/or pH indicators as described above.

In some embodiments, the present application provides an AST kit in a fresh or lyophilized format, with media as described herein, TB drugs, microtiter plates, microtiter plates pre-coated with the media components described herein, tubes, including tubes pre-coated with the media components described herein, and/or conventional devices for carrying our antibiotic susceptibility testing.

In another aspect, the present application provides a method for screening a plurality of test compounds or drugs for their ability to inhibit the growth of M. tuberculosis. In one embodiment, the method comprises the steps of (a) inoculating a culture medium comprising a nutrient composition described in the present application with a M. tuberculosis isolate to form a control culture; (b) inoculating in parallel a plurality of secondary cultures comprising the M. tuberculosis in step (a) to from a plurality of test cultures, wherein each of the plurality of test cultures comprises a different test compound in the medium comprising the composition in step (a); (c) incubating in parallel the first culture and the test cultures under neutral pH conditions for a period of time and temperature sufficient to observe growth of the control sample in the absence of the different test compounds in step (b); (d) comparing the growth of the M. tuberculosis isolate in the presence or absence of the test compound; and (e) determining whether one or more of the test compounds inhibit growth of the M. tuberculosis isolate, based on the comparison in step (d).

Generally, the in vitro drug screening method proceeds through three steps: performing the screening assay to identify anti-TB activity; preforming a secondary test to confirm it; and determination of the minimal inhibitory concentration for hit using the broth microdilution method.

The drug screening method may include test compounds distributed in any suitable plate design known to those skilled in the art. In preferred embodiments, a microtiter plate is used for drug screening. Exemplary microtiter plates include, e.g., 6, 12, 24, 48, 96, 384 or 1536-well formats. In some embodiment, a microplate may be manufactured with 3456 or 9600 wells, including an “array tape” product providing a continuous strip of microplates embossed on a flexible plastic tape.

The method may utilize any M. tuberculosis strain or isolate, including both antibiotic-sensitive or antibiotic-resistant strains. The strain or isolate may be selected on the basis of growth characteristics, media composition, antibiotic resistance, and the like. Further, the method may be carried out using any one of a number of different nutrient compositions or culture conditions as described herein. For example, in certain embodiments, the drug screening may be carried out using an M. tuberculosis strain or isolate grown in a particular medium composition defined by a particular primary nitrogen source (e.g., type of amino acid(s)), primary carbon source, and/or concentration thereof.

In some embodiment, the drug screening may be carried out in parallel screening panels (e.g., microtiter plates), where each panel includes a strain or isolate grown in a particular medium composition differing with respect to, for example, the primary nitrogen source (e.g., type of amino acid(s)), the primary carbon source, or the concentration of one or components. Alternatively, each parallel panel may contain a different strain or isolate of M. tuberculosis.

In certain preferred embodiments, the test compounds are distributed into microtiter plates containing the nutrient compositions of the present application cultured under neutral pH conditions. Preferably, the test compounds are obtained from suitable sources in a pre-plated format.

The plurality of test compounds may include hundreds, thousands and millions of test compounds publicly available from a variety of sources known in the art. Exemplary compound libraries may be obtained from a number of sources known to those skilled in the art, including pre-plated microtiter plates, such as those from e.g., the National Institutes of Health via the Developmental Therapeutics Program (DTP), including the NCI Diversity Set VI (˜3,000 compounds), the Mechanistic Set VI of 811 compounds, the Approved Oncology Drugs set X, the Mechanistic Set VI of 811 compounds, the Natural Products Set V of compounds; and a variety of other compound libraries from Apexbio at www.apexbt.com; Charles River www.criver.com, and many others.

In some embodiments, variant test compounds or libraries thereof are generated, based on the chemical structures corresponding those identified in the screen as “hits”. In some embodiments, the variant compounds are designed, based on 3D computer modeling or artificial intelligence.

In some embodiments, the test compounds found to inhibit growth of M. tuberculosis are incubated in the presence or absence of different test compound concentrations, where a MIC is determined for each test compounds under evaluation. In this case, the MIC corresponds to the lowest concentration of the test compound inhibiting visible growth of M. tuberculosis.

In another aspect, the present application provides a pharmaceutical composition for treatment or prevention of tuberculosis comprising a compound set forth in Table 12 in combination with a pharmaceutically acceptable carrier.

In another aspect, the present application provides a method for treatment of M. tuberculosis, comprising administering to a subject in need thereof a compound set forth in Table 12 in combination with a pharmaceutically acceptable carrier and other TB drugs.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bacterial levels, among others. The dosage can be determined by a physician and adjusted, as necessary, to suit observed effects of the treatment.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the pharmaceutical composition can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like.

In some embodiments, the drug is administered in a controlled-release formulation. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

The methods described herein can further comprise administering a second agent and/or treatment to the subject, e.g., as part of a combinatorial therapy. By way of non-limiting example, a subject with tuberculosis can be further administered an antibiotic or a subject likely to experience transplant rejection can be further administered an immunosuppressant.

In some embodiments, a subject is administered a second anti-tuberculosis agent. Exemplary secondary anti-tuberculosis agents for administration include, but are not limited to pyrazinamide, rifampin, rifapentine, isoniazid, ethambutol, bedaquiline, linezolid, moxifloxacin, delamanid, pretomanid, amikacin, clofazimine, cycloserine, rifabutin, levofloxacin, ethionamide, p-aminosalicylic acid, streptornycin, fluoroquinolone, and combinations thereof.

In certain embodiments, an effective dose of one or more active agents or drugs as described herein can be administered to a patient once. In certain embodiments, an effective dose of a composition can be administered to a patient repeatedly. For systemic administration, subjects can be administered a therapeutic amount of a composition, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., bacterial counts by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of the compositions described herein, according to the methods described herein depend upon, for example, the form of the composition, its potency, and the extent to which symptoms, markers, or indicators of a condition described herein are desired to be reduced, for example the percentage reduction desired for a symptom or the extent to which, for example, successful immune responses are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g., the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., bacterial levels. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of tuberculosis. When using an experimental animal model, efficacy of treatment is evidenced when a statistically significant change in a marker is observed, e.g., a decreased in the presence of Mycobacterium in the subject, or a progression towards latent instead of active disease.

In vitro and animal model assays are provided herein which allow the assessment of a given dose of a composition. By way of non-limiting example, the effects of a dose of a composition can be assessed by administering the composition to an animal model of tuberculosis, e.g., a mouse infected with Mycobacterium.

### EXAMPLES

Materials and Methods

Strains and Media

M. tuberculosis H37Ra was grown on Middlebrook 7H11 agar plates or in 7H9 broth supplemented with 0.5% (v/v) glycerol and 10% (v/v) albumin-dextrose-catalase (ADC).

Unless otherwise noted, 4 different types of growth media were utilized in the experiments described below
(1) PZA-S1-Plus I: PZA-S1-Minimal supplemented with one or more amino acid with various amount as primary nitrogen source(s) and further comprising: 0.5 g/L citric acid, 10 ml/L glycerol, and 0.0125% Tween 80, with or without 20 μg/ml phenol red, where the pH was adjusted to 6.8 with 1 M NaOH.
(2) PZA-S1-Plus II: PZA-S1-Minimal supplemented with one or more amino acids as primary nitrogen source(s) and further comprising: 0.5 g/L citric acid, 10 ml/L glycerol, no Tween 80 or ADC, with or without 20 μg/ml phenol red, where the pH was adjusted to 6.8 with 1 M NaOH.
(3) PZA-S1-Plus III: PZA-S1-Minimal supplemented with one or more lipids as primary carbon source(s) and further comprising: 0.25 g/L ammonium chloride, with or without Tween 80 and ADC (0.5 g/L albumin, 0.2 g/L dextrose 0.0003 g/L Catalase), and with or without 20 μg/ml phenol red where the pH was adjusted to 6.8 with 1 M NaOH.
(4) PZA-S1-Plus IV: PZA-S1-Minimal supplemented with 20 μg/ml phenol red and (i) one or more amino acids as primary nitrogen source(s) and 0.0125% Tween 80 and ADC; or (ii) one or more amino acids as primary nitrogen source(s), one or more carbon source(s), and 0.5 g/L albumin, 0.2 g/L dextrose 0.0003 g/L Catalases. The pH in the media was adjusted to 6.8 with 1 M NaOH.

For primary carbon source media experiments, the defined media contained PZA-S1-Minimal supplemented with 0.25 g/L ammonium chloride, and either cholesterol, glycerol, butyric acid, or lactic acid at various concentration as described in the experiments below.

For primary nitrogen source media experiments, the defined media contained PZA-S1-Minimal supplemented with 0.5 g/L citric acid, 10 ml/L glycerol, and either 2 g/L amino acid or an amino acid mixture as described in the experiments below.

When using ammonium salts as the primary nitrogen source, the defined media contained PZA-S1-Minimal supplemented with 0.5 g/L citric acid, 10 ml/L glycerol and 0.25 g/L of an ammonium salt as described in the experiments below.

The pH of each growth medium was adjusted to 6.8 with 1 M sodium hydroxide or 1 N hydrochloric acid, unless otherwise noted. For solid media, 1.5% (w/v) Bacto agar was added to the medium. To avoid contamination, an antibiotic cocktail containing cycloheximide (10 μg/ml), carbenicillin (50 μg/ml), polymyxin B (25 μg/ml), and trimethoprim (20 μg/ml), was added, as needed. Where dextrose and/or albumin were added, the concentrations are as described as set forth below.

M. tuberculosis H37Ra and PZA resistant strains used in the following experiments are described herein. M. tuberculosis H37Ra (attenuated) and M. tuberculosis H37Rv (virulent) were chosen, because these strains predicted MICs of clinical isolates equally well, based on clinical susceptibility data and because H37Ra has the benefit of lower experimental costs and less administrative barriers with regard to biosafety Level III restrictions. Additional resistant strains are described in the Examples.

As further described below, PZA experiments were carried using PZA-S1-Plus I or II or III or IV media in one or four formats (unless otherwise noted):
(1) Microtiter plate format in liquid medium (air exchange) (PZA-S1-Plus I, II III and IV);
(2) Liquid media in large test tube (15 ml) (more air) (PZA-S1-Plus I, II, III and IV);
(3) Liquid media in small tube (200 μl) (less air or different air exchange rate between (2) and (3) (PZA-S1-Plus I, II, III and IV); and
(4) Solid media (agar plate) (PZA-S1-Plus I, II, III or IV with addition 1.5% agar and ADC (0.5 g/L albumin, 0.2 g/L dextrose 0.0003 g/L Catalase).

Broth microdilution method for determining MIC of PZA with liquid medium in microtiter plates, tubes, or other devices.

M. tuberculosis H37Ra from frozen stocks were grown on Middlebrook 7H11 agar plates supplemented with 0.5% (v/v) glycerol and 10% (v/v) albumin-dextrose-catalase (ADC) at 37° C. for 3˜4 weeks. In exemplary embodiments, two-fold serial dilutions of PZA were prepared for adding successively larger amounts of PZA to microtiter wells between 12.5 and 800 μg/ml. A set of media with PZA and without PZA (control) was used in each assay. At least six colonies of M. tuberculosis were scraped from solid agar plates using a sterile inoculating loop and transferred into the related liquid medium, 1×PBS (pH 7.2), or sterile water with 10 glass 0.5 mm beads. In some instances, inocula were derived 2-3 week cultures grown in 7H9 media. Samples were vortexed for about 30-60 s and allowed to settle for 15 min to remove clumps.

The supernatant suspensions were diluted in media to provide 0.5 McFarland units of cells, standardized using the McFarland Densitometer according to the manufacturer's instructions. This unit of cells corresponds to the lowest number that is readily detectable (either by eye or by turbidimetric measurements). The bacterial suspensions are then visually compared to the McFarland standards estimating the bacterial density. The density (live cell colony-forming unit, CFU/ml) varies in different bacteria.

A 0.5 McFarland standard suspension corresponds to approximately 10⁷ CFU/ml for M. tuberculosis and M. kansasii and 10⁸ CFU/ml for other mycobacteria. In this case, 0.5 McFarland units correspond to ˜1×10⁷ CFU/ml, which are then diluted 1:50 and 1:100 with the PZA-S1-Minimal medium. The standard inoculum size in each well was ˜1×10⁵ CFU/ml to ˜5×10⁵ CFU/ml. There is a good concordance between the McFarland scale and the CFU/ml for M. tuberculosis. In these experiments, 0.2 ml of the bacterial suspension was added to each well in a 96 well plate (or tube) as described. A control set of dilutions without PZA were included in each microtiter plate. The microtiter plates were sealed with parafilm to avoid drying and were incubated at 37° C. MIC results from these experiments correspond to the lowest concentrations of drug inhibiting visible growth in liquid media at one, two, three, four or six weeks of exposure. The first MIC result was recorded and considered as the relevant test result. Each test was conducted in duplicate or triplicate.

The microtiter plates were sealed with parafilm and incubated at 37° C. The MICs were determined on the basis of the lowest PZA concentration inhibiting growth in liquid medium at one, two, three, four or six weeks of exposure.

Susceptibility of PZA on Agar Plates

Fresh colonies were taken from 7H111 agar plates and suspended in 1×PBS (pH 7.2). The turbidity was adjusted to 0.5 McFarland unit (corresponding to 1 to 5×10⁷ CFU/ml). 20 ml of 1.5% (w/v) agar media was poured in 100 mm petri dish (i.e., 5 ml in each quadrant). After the agar plate solidified, 50 μl of M. tuberculosis inoculum was spread on each quadrant of the petri dish using sterile 5 mm diameter glass beads or a sterile cotton applicator. After the surface of agar plate was dry, paper discs with different amounts of PZA were disposed on the agar plate. After being sealed in plastic bags, the plates were inverted and placed in a 37° C. incubator for 3-4 weeks. The plates were observed for zones of clearing after 2, 3 and 4 weeks.

For the agar proportion method, a drug susceptibility test was carried out according to the instructions of the Clinical and Laboratory Standards Institute. Serial dilutions were prepared. The PZA-containing and drug-free control agar plates were inoculated with 50 μl of a M. tuberculosis suspension. The drug-free control plates were inoculated with a 1:100 dilution of the M. tuberculosis suspension plated on the PZA-containing plates. PZA concentrations in the media were either 0, 25, 50, 100, 200, 400 or 800 μg/ml. The inoculated culture media were incubated at 37° C. for 4 weeks. Plates cultured in drug-free media having between 50 to 150 colonies per quadrant on the agar plate, were compared to the number of colonies in the corresponding PZA-containing plates. The proportion of PZA resistant M. tuberculosis in the plates was determined and expressed as a percentage of the total population of M. tuberculosis plated on the drug-containing plates. Where there are more colonies on the drug plate than the no drug control plate, the M. tuberculosis isolate is considered “resistant” (≥1%).

Determination of Culture pH.

To determine the pH changes in the cultures, a phenol red indicator was included at a concentration of 15 μg/ml. Media at pH values between 6.0 to 8.5 were used as standards for comparison to the culture plates for determining pH.

PZA-Like Drug Screening

Compounds for PZA susceptibility drug screening were obtained from the National Cancer Institute (NCI), National Institutes of Health (NIH) according to the Developmental Therapeutics Program (DTP) were prepared for use in the drug screens described below as stock solutions in dimethyl sulfoxide (DMSO) at a concentration of 10 mM in 96 or 384-well plates stored at −20° C. The concentration of each drug in the assay was 100 μM. Control wells contained an equivalent amount of DMSO.

M. tuberculosis suspensions (at ˜5×10⁵ CFU/ml) were plated at 200 μl/well in 96 well plates or 100 μl/well in 384-well plate in the presence of PZA in different media at pH 6.8, including 7H9; PZA-S1 (PZA-S1-Minimal supplemented with 2.0 g/L L-Alanine as primary nitrogen source, 10 ml/L glycerol and 0.5 g/L citric acid); SLac (PZA-S1-Minimal supplemented with 0.25 g/L ammonium chloride, 1.0 g/L lactate); or SBA (PZA-S1-Minimal supplemented with 0.25 g/L ammonium chloride, 1.0 g/L butyric acid as primary carbon source) and incubated at 37° C. After 3 weeks, viability was visually examined. Prospective “hits” were identified as compounds inhibiting growth of M. tuberculosis in PZA-S or PZA-S plus media, but not in conventional culture media, such as 7H9. Cultures from prospective “hits” were dispensed into 96 well plates for the secondary evaluation in 7H9 media and the other three defined media. Confirmed hits were selected for MIC assay determination by the broth microdilution assay. The MIC value was based on identification of the lowest concentration of a drug capable of causing no visible growth in a well compared to its corresponding no drug control.

### Example 1. Growth of Cells and Activity of PZA in Broth Media with a Primary Carbon Source at Neutral pH

M. tuberculosis was grown on a defined medium as described below. Glycerol, citric acid, cholesterol, fatty acid (butyric acid), and lactate were utilized as primary carbon sources for M. tuberculosis growth. The defined media contained PZA-S1-Minimal, 0.25 g/L ammonium chloride. The media contained glycerol, citric acid, cholesterol, sodium butyrate or sodium lactate in the concentrations set forth in Table 1 below. MIC values for PZA were determined by the broth microdilution method following 20 days of growth at 37° C. The degree of growth was based on the density of the bacilli suspension (+; ++; +++).

As shown in Table 1, PZA was active at inhibiting M. tuberculosis growth at neutral pH 6.8 in defined media containing cholesterol, citric acid, glycerol, butyric acid, lactate or acetate as the primary carbon source and ammonium chloride as the primary nitrogen source.

TABLE 1
Effect of cholesterol, glycerol, sodium butyrate or sodium
lactate as primary carbon sources on M. tuberculosis growth
and PZA susceptibility at neutral pH 6.8 in vitro.

Carbon source in

| defined medium* | Concentration | | Growth | MIC of PZA (μg/ml) |
|---|---|---|---|---|
| Cholesterol | 160 | mg/L | ++ | 50 |
| | 80 | mg/L | +++ | 400 |
| | 40 | mg/L | +++ | 400 |
| Glycerol | 10 | ml/L | ++ | 100 |
| | 20 | ml/L | ++ | 100 |
| Sodium butyrate | 1 | g/L | ++ | 100 |
| | 0.5 | g/L | +++ | 200 |
| Sodium lactate | 1 | g/L | ++ | 200 |
| | 0.5 | g/L | + | 50 |

*containing PZA-S1-Minimal, 0.25 g/L ammonium chloride and one primary carbon source.

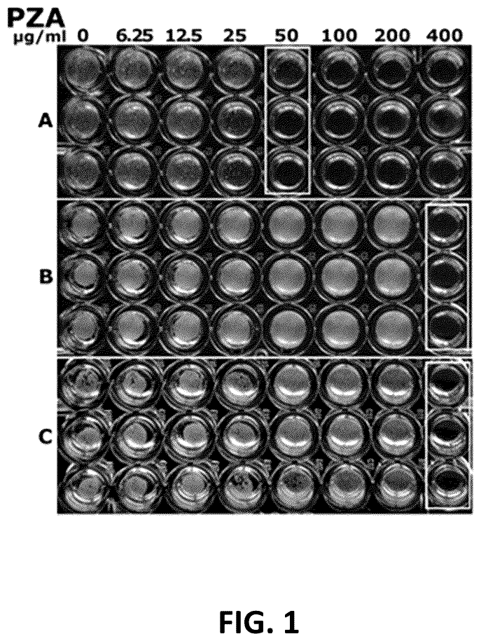

At a cholesterol concentration of 160 mg/ml, the MIC for PZA was 50 μg/ml. At lower cholesterol concentrations of 80 mg/L and 40 mg/L, the MIC was 400 μg/ml (Table 1 and FIG. 1). In contrast, a lower MIC of 50 μg/L was observed at a lactate concentration of 0.5 g/L, while a higher MIC of 200 μg/ml was observed at a lactate concentration of 1 g/L. The MICs of PZA at butyric acid concentrations between 0.5 g/L and 1 g/L and at ammonium acetate concentrations between 0.25 to 4 g/L were between about 100 μg/ml and 200 μg/ml. (see FIG. 1, Table 1, and Table 3). However, growth was greatly inhibited when lactate and butyric acid were above 2 g/L in the medium. Moreover, less growth was observed in the control without PZA at a cholesterol concentration of 160 mg/L than at cholesterol concentrations of 80 mg/L and 40 mg/L (Table 1 and FIG. 1).

### Example 2. M. tuberculosis Growth and PZA Activity in Broth Media with Different Amino Acids as Primary Nitrogen Sources at Neutral pH

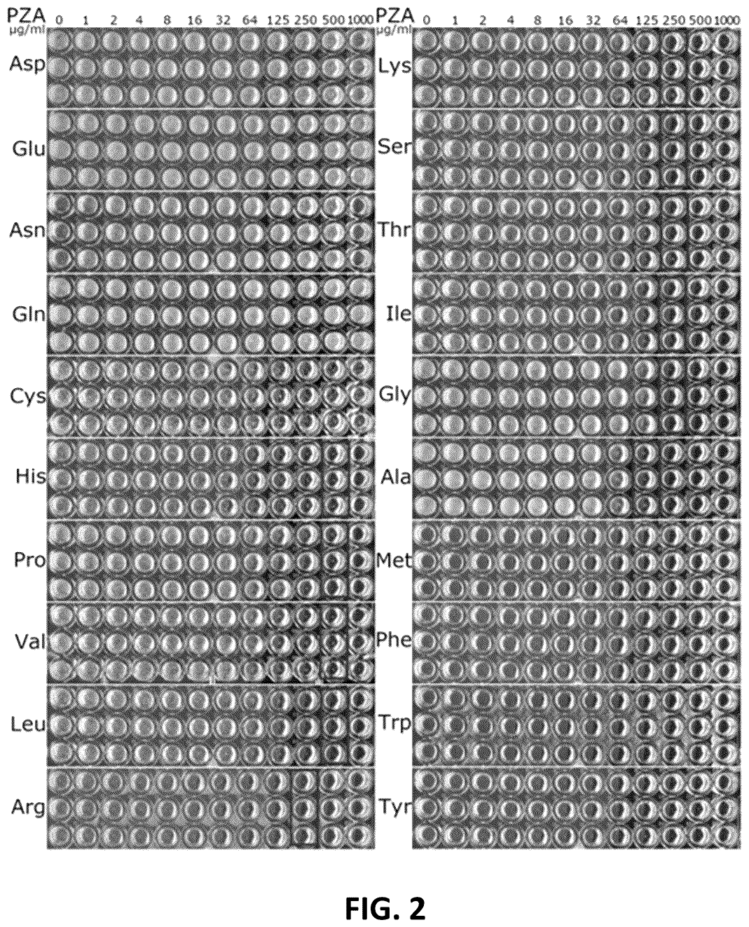

Growth and PZA susceptibility of M. tuberculosis was evaluated under neutral pH conditions in the presence of 20 different amino acids as primary nitrogen sources. In these experiments, M. tuberculosis was grown in a defined medium containing 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, and one of 20 amino acids in the amounts indicated in Table 2. The MIC of PZA for each amino acid culture was determined at 37° C. for 20 days by the broth microdilution method. The results of this assay are shown in FIG. 2. The MIC of PZA was determined at 37° C. following a 20-day incubation at 37° C. via the broth microdilution method utilizing a 96 well plate. The MIC with the lowest growth inhibition of PZA concentration in the well and is labeled in FIG. 2 with a box encircling the wells corresponding to that PZA concentration.

After 20 days of incubation at 37° C., PZA susceptibility results were determined based on visual inspection. As shown in FIG. 2 and Table 2, cultures were found to be susceptible to PZA at pH 6.8 for each of 11 different amino acids in the defined medium (i.e., 500 μg/ml or less). More specifically, the MIC of PZA was determined to be 500 μg/ml in L-Histidine, L-Proline, L-Valine or L-Leucine-containing defined media; 250 μg/ml in L-Arginine, L-Lysine, L-Serine, L-Threonine, L-Isoleucine or Glycine-containing defined media; and 125 μg/ml in PZA-S1 media. No growth was observed in defined media containing L-Methionine, L-Phenylalanine, L-Tryptophan or L-Tyrosine as the primary nitrogen source; consequently, the MIC of PZA was not applicable to these cultures.

As shown in FIG. 2 and Table 2, the extent of M. tuberculosis growth for a given amino acid did not generally correlate with the MIC of PZA grown using the same amino acid as the primary nitrogen source. For example, while abundant growth (+++) was observed in media containing L-Aspartic acid, L-Glutamic acid, L-Asparagine, L-Glutamine, L-Valine, Glycine and L-Alanine, PZA susceptibility was poor in culture individually grown with L-Aspartic acid, L-Glutamic acid, L-Asparagine, and L-Glutamine (i.e., MIC>1000 μg/ml or undetectable). There was moderate growth in the other amino acid-containing defined media. For cultures individually grown with L-Asparagine, L-Aspartic acid, L-Glutamine or L-Glutamic acid, PZA activity did not change when the PZA concentration was increased to 5 mg/ml in the media in microtiter plate or when their amino acid concentrations were decreased to 0.1 g/L in the media. Moreover, for the 11 amino acids where PZA was active in the media, PZA susceptibility or growth was not significantly different at amino acid concentrations of 4 g/L or 0.5 g/L.

For example, in the medium with L-Ala as the nitrogen source, the MIC of PZA showed no changes when the concentration of L-Ala was in the range from 4 to 0.5 g/L at pH 6.8 in the minimal media in microtiter plate (Table 2). The growth rate did not change significantly due to the change in the concentration of either L-Asn or L-Ala as the nitrogen source in the media. Since the growth rates between L-Asn and L-Ala (as the nitrogen sources in the media) were similar, this indicated that the activity of PZA was unlikely to be correlated with the state of growth of M. tuberculosis but, instead, with the type of nutrient in the culture media. Growth in the presence of L-Alanine was the most effective in terms of growth (+++) and PZA susceptibility. Therefore, PZA-S1 medium containing L-Alanine and glycerol are used herein with reference to a “PZA-S1” media composition, which was chosen for use in liquid broth or agar plate experiments described herein, unless otherwise noted.

In conventional PZA susceptibility testing conducted under acidic conditions, L-Asn, L-Glu, L-Gln or L-Asp have been used as the primary nitrogen source. These same amino acids supported the highest levels of M. tuberculosis growth under neutral pH conditions. However, the data surprisingly showed that in the neutral pH conditions examined herein, PZA was not active in culture media containing these amino acids (i.e., MIC>1000 g/ml), despite their reliance and use in conventional PZA susceptibility testing conducted under acidic conditions.

TABLE 2
PZA susceptibility and growth of M. tuberculosis at neutral
pH 6.8 using 20 different amino acids as primary nitrogen
sources and using glycerol as the primary carbon source
in defined media (containing PZA-S1-Minimal, 10 ml/L glycerol,
0.5 g/L citric acid plus one amino acid).

| Amino acid in defined | | | |
|---|---|---|---|
| medium as primary | Concentration | MIC of PZA | |
| nitrogen source | (g/L) | (μg/ml) | Growth* |
| L-Aspartic acid (Asp) | 2.0 | >1000 | +++ |
| L-Glutamic acid (Glu) | 2.0 | >1000 | +++ |
| L-Asparagine (Asn) | 2.0 | >1000 | +++ |
| | 0.5 | >1000 | +++ |
| | 0.1 | >1000 | +++ |
| L-Glutamine (Gln) | 2.0 | >1000 | +++ |
| L-Cysteine (Cys) | 2.0 | >1000 | ++ |
| L-Histidine (His) | 2.0 | 500 | ++ |
| L-Proline (Pro) | 2.0 | 500 | ++ |
| L-Valine (Val) | 2.0 | 500 | +++ |
| L-Leucine (Leu) | 2.0 | 500 | ++ |
| L-Arginine (Arg) | 2.0 | 250 | + |
| L-Lysine (Lys) | 2.0 | 250 | + |
| L-Serine (Ser) | 2.0 | 250 | + |
| L-Threonine (Thr) | 2.0 | 250 | + |
| L-Isoleucine (Ile) | 2.0 | 250 | ++ |
| Glycine (Gly) | 2.0 | 250 | +++ |
| L-Alanine (Ala) | 4.0 | 125 | +++ |
| | 2.0 | 125 | |
| | 1.0 | 125 | +++ |
| | 0.5 | 125 | +++ |
| L-Methionine (Met) | 2.0 | ND | − |
| L-Phenylalanine (Phe) | 2.0 | ND | − |
| L-Tryptophan (Trp) | 2.0 | ND | − |
| L-Tyrosine* (Tyr) | 2.0 | ND | − |

*Amount of growth in the medium without PZA (first column of each sample in FIG. 1) The amount of growth could be estimated from the visible density of the M. tuberculosis suspension
(“−” no growth; “+” visible growth; “++” visible growth; “+++” abundant growth).
** Partially dissolved; insoluble portion removed before adjusting the media pH; ND, not detected.

### Example 3. M. tuberculosis Growth and PZA Activity in Defined Broth Media with Different Ammonium Salts as the Primary Nitrogen Source

The effect of 8 different ammonium salts as primary nitrogen sources as a function of inoculum size was evaluated with regard to growth and PZA susceptibility under neutral growth conditions (pH 6.8) in defined medium containing PZA-S1-Minimal, 10 ml/L glycerol, 0.5 g/L citric acid and 0.25 g/L ammonium salt (Table 3). The MICs for PZA was determined after 3 weeks and 5 weeks of growth at 37° C. by the broth microdilution method.

TABLE 3
Effect of ammonium salts as primary nitrogen sources and inoculum sizes on growth and
PZA susceptibility of M. tuberculosis H37Ra by the broth microdilution method.

| Time point | | | 3 weeks | | | | 5 weeks | | | |
|---|---|---|---|---|---|---|---|---|---|---|
| Dilution from 0.5 McFarland unit | | | 1/10 | 1/25 | 1/50 | 1/100 | 1/10 | 1/25 | 1/50 | 1/100 |
| Media* | Ammonium | MIC* | 400 | 200 | 200 | 200 | 400 | 400 | 400 | 200 |
| | acetate | Growth | ++++ | +++ | ++ | ++ | ++++ | +++ | ++ | ++ |
| | Ammonium | MIC* | 100 | 50 | − | − | 200 | 100 | 50 | 50 |
| | chloride | Growth | ++ | + | − | − | ++++ | +++ | ++ | + |
| | Ammonium | MIC* | 800 | 400 | 100 | − | 800 | 800 | 400 | 200 |
| | succinate | Growth | ++ | + | + | − | +++ | ++ | ++ | ++ |
| | Ammonium | MIC* | 100 | − | − | − | 400 | 200 | 100 | 50 |
| | citrate dibasic | Growth | + | − | − | − | +++ | ++ | + | + |
| | Ammonium | MIC* | 100 | − | − | − | 200 | 100 | 50 | 25 |
| | tartrate dibasic | Growth | + | − | − | − | +++ | ++ | + | + |
| | Ammonium | MIC* | 50 | − | − | − | 100 | 50 | 25 | 25 |
| | formate | Growth | + | − | − | − | +++ | ++ | + | + |
| | Ammonium | MIC* | 50 | − | − | − | 100 | 50 | 25 | 25 |
| | nitrate | Growth | + | − | − | − | +++ | ++ | + | + |
| | Ammonium | MIC* | − | − | − | − | 25 | ≤12.5 | − | − |
| | bicarbonate | Growth | − | − | − | − | ++ | + | − | − |

*containing PZA-S1-Minimal, 10 ml/L glycerol, 0.5 g/L citric acid plus one ammonium salt as primary nitrogen source

As shown in Table 3, M. tuberculosis growth was most abundant in defined media containing ammonium acetate at both 3 and 5 weeks of incubation at 37° C. By comparison, cell cultures containing the other ammonium salts grew slowly, however, growth was visible after five weeks of cultivation in all eight media. The MIC of PZA varied depending on the inoculum size and the ammonium salt. There was a 2-8 fold increase in MIC between an 1/100 and 1/10 inoculum size dilutions from a 0.5 McFarland unit starting point. Thus, a higher MIC correlated with a larger inoculum. With a 1/100 dilution of 0.5 McFarland unit, the MIC of PZA after 5 weeks of growth was 200 μg/ml when ammonium acetate or ammonium succinate was used as the primary nitrogen source; 50 μg/ml when ammonium chloride or ammonium citrate dibasic was used as the primary nitrogen source; 25 μg/ml when ammonium tartrate dibasic, ammonium formate, or ammonium nitrate was used as the primary nitrogen source. No growth was observed after 3 weeks when ammonium bicarbonate was the primary nitrogen source. However, after 5 weeks of growth, a MIC of PZA activity was observed at 25 μg/ml with a 1/10 dilution at 0.5 McFarland unit. The MIC of PZA was at 50 μg/ml in ammonium chloride and ammonium acetate defined media at 4 or 8 g/L concentration with slower growth compared at 0.25 g/L concentration.

### Example 4. PZA Susceptibility on Agar Plates at Neutral pH

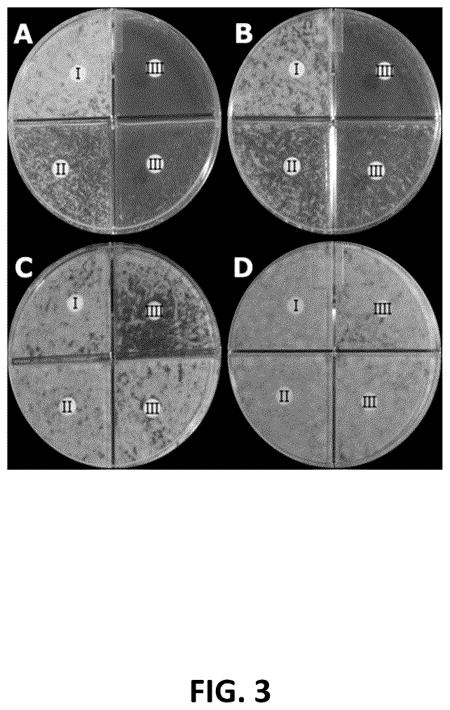

To confirm whether PZA inhibition could be obtained on agar plates at neutral pH 6.8, M. tuberculosis H37Ra was spread onto PZA-S1 or PZA-S1-plus agar (1.5% w/v) plates additionally supplemented with ADC (0.5 g/L albumin, 0.2 g/L dextrose and 0.3 mg/L catalase) and different primary nitrogen sources, specifically, PZA-S1 medium (FIG. 3, panel A); PZA-S1-Plus NC medium with ammonium chloride as the primary nitrogen source (FIG. 3, panel B) or PZA-S1-Plus NA with ammonium acetate as the primary nitrogen source (FIG. 3, panel C). M. tuberculosis H37Ra was additionally seeded onto a 7H11 agar plate supplemented with ADC (0.5 g/L albumin, 0.2 g/L dextrose and 0.3 mg/L catalase). Hereinafter, the medium in FIG. 3, panel A is referred to as PZA-S1-Plus medium. ADC was added in view of the knowledge that agar contains long-chain fatty acids and lipids, which have an inhibitory effect on the growth of M. tuberculosis and that albumin has been shown to relieve the inhibitory effect against M. tuberculosis growth on agar plates.

In the PZA-S1 or PZA-S1-Plus agar plates supplemented with albumin at a concentration of 0.5 g/L, equivalent growth was observed in each control plate lacking PZA (FIG. 3, quadrant I in panels, A, B, C and D). In contrast, PZA inhibited lawn formation of M. tuberculosis on neutral pH plates where a paper disc containing 400, 800 and 1600 μg PZA was added to quadrants II, III and 1III on each of the plates in FIG. 3, panels A, B, and C. However, no inhibition (full lawn of cells) was observed on the 7H11 agar plate (pH 6.6±0.2) in panel D. The antibiotic susceptibility test on PZA-S1 agar plates enables use of the agar proportion method as described in Example 11 below.

### Example 5. Identification of PZA Resistant M. tuberculosis Strains in Liquid Broth or on Agar Plates

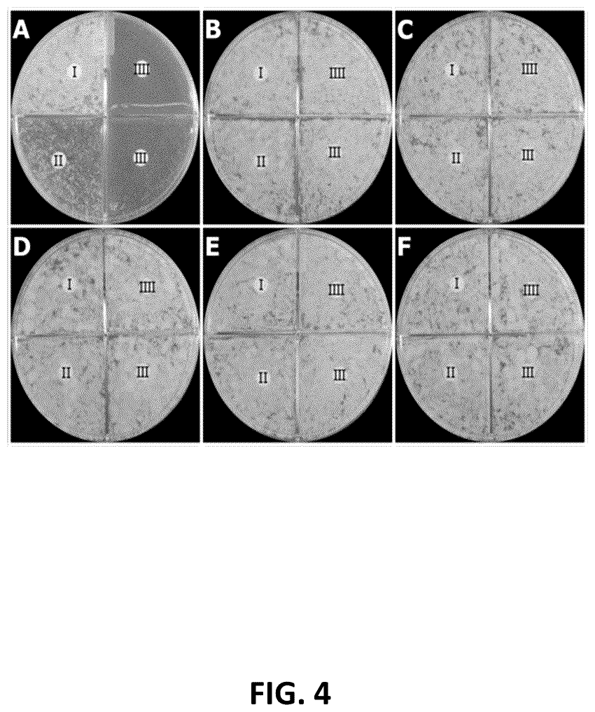

With a knowledge of the conditions in which PZA is active in neutral pH conditions both in broth media and on agar plates, PZA susceptibility tests were carried out to confirm M. tuberculosis PZA resistance in five known PZA resistant and control strains. These PZA susceptibility tests were carried out at pH 6.8 in broth media (Table 4) and on agar plates (FIG. 4). These tests employed PZA-S1-Plus broth cultures (pH 6.8) supplemented with ammonium chloride (NH₄Cl, 0.25 g/L) (PZA-S1-Plus NC), L-Alanine (L-Ala, 2 g/L)(PZA-S1), or ammonium acetate (NH₄AOc, 0.25 g/L) (PZA-S1-Plus NA) as the primary nitrogen sources and 1% glycerol as the primary carbon source. The PZA-sensitive H37Ra strain and the five PZA-resistant H37Ra strains were grown in the presence of PZA for 3 weeks by the broth microdilution method. As shown in Table 4, the MIC for PZA to inhibit the growth of M. tuberculosis H37Ra was 100, 200 and 200 μg/ml PZA in ammonium chloride, L-Alanine, and ammonium acetate media, respectively. In contrast, the MIC for PZA was greater than 800 μg/ml against the five PZA resistant M. tuberculosis strains.

TABLE 4
PZA Susceptibility of M. tuberculosis H37Ra and five PZA resistant strains at
pH 6.8 in broth media at 37° C. for 3 weeks by the broth microdilution method.

| | Media and MIC of PZA (μg/ml) | | |
|---|---|---|---|
| Strain | PZA-S1-Plus NC | PZA-S1 | PZA-S1-Plus NA |
| M. tuberculosis H37Ra | 100 | 200 | 200 |
| M. tuberculosis H37Ra PZA^{R} #6 | >800 | >800 | >800 |
| M. tuberculosis H37Ra PZA^{R} #78 | >800 | >800 | >800 |
| M. tuberculosis H37Ra PZA^{R} #98 | >800 | >800 | >800 |
| M. tuberculosis H37Ra PZA^{R} #106 | >800 | >800 | >800 |
| M. tuberculosis H37Ra PZA^{R} #186 | >800 | >800 | >800 |

The PZA susceptibility tests were carried out on PZA-S1-Plus agar media at neutral pH 6.8 using the paper disk method. For the PZA susceptible strain H37Ra, there was a small inhibition zone with 400 μg PZA (FIG. 4, panel A, quadrant II) and strong growth inhibition with 800 μg and 1600 μg PZA (FIG. 4, panel A, quadrants III and IIII). In contrast, no inhibition was observed in the absence of PZA (FIG. 4, all quadrant I areas in panels A-F), or in the presence of PZA for the PZA-resistant strains (FIG. 4, panels B-F, all quadrants). Thus, PZA resistance of M. tuberculosis can be identified in both defined media and on solid agar plates. It should be noted that the use of paper disks can be similarly used in an agar proportion method as further described in Example 11 below.

### Example 6. PZA Susceptibility Effect on pH in Lactate or L-Alanine Defined Broth Media

To investigate the effects of pH on PZA susceptibility in the defined media, PZA susceptibility of M. tuberculosis was tested at a pH range between 5.0 to 8.5 in defined media SLac supplemented with lactate as the primary carbon source (Table 5) or PZA-S1 medium with L-Alanine as the primary nitrogen source and glycerol as the primary carbon source (Table 6). Two sets of liquid cultures containing the foregoing media compositions were inoculated with 1/100 dilutions of a 0.5 McFarland unit culture of M. tuberculosis H37Ra. PZA activity was evidenced by macroscopically visible bacterial growth following a two week incubation at 37° C.

The results in Tables 5 and 6 show that both growth and PZA susceptibility are significantly affected by pH. In alkaline (pH 7.5, 8.0, and 8.5) and acidic conditions (pH 5.0), no visible growth was seen in the presence or absence of PZA when lactate was used as the primary carbon source (Table 5). In neutral pH 7.0 conditions, PZA activity was detectable, but M. tuberculosis failed to grow in PZA concentrations beyond 200 μg/ml for both the lactate (Table 5) and L-Alanine/glycerol samples (Table 6). Further, an equivalent MIC for PZA of 200 μg/ml was obtained at pH 8.0 to 6.5 for the L-Alanine/glycerol samples (Table 6). However, as shown in Table 6, the MIC for PZA against M. tuberculosis (200 μg/ml) for the L-Alanine/glycerol samples was 8-fold lower in acidic conditions (pH 6.5 and pH 6.0) than in neutral pH 7.0 conditions (25 μg/ml). Further, at pH 5.5, the MIC for PZA against M. tuberculosis in the lactate samples was less than 12.5 μg/ml, while the MIC was 100 μg/ml at pH 6.0 and 25 μg/ml at pH 5.5 with the L-Alanine/glycerol samples. These results reflect a lower MIC for PZA against M. tuberculosis in acidic pH conditions than in neutral pH conditions. The results are consistent with PZA being active at neutral pH in vitro and more active at acidic pH in vivo.

TABLE 5
Activity of PZA against M. tuberculosis H37Ra in
SLac broth medium containing lactate as primary carbon
source under pH conditions from 5.0 to 8.5.

| | PZA (μg/ml) | | | | | | | |
|---|---|---|---|---|---|---|---|---|
| pH | 0 | 12.5 | 25 | 50 | 100 | 200 | 400 | 800 |
| 8.5 | − | − | − | − | − | − | − | − |
| 8.0 | − | − | − | − | − | − | − | − |
| 7.5 | − | − | − | − | − | − | − | − |
| 7.0 | + | + | + | + | + | − | − | − |
| 6.5 | + | + | − | − | − | − | − | − |
| 6.0 | + | + | − | − | − | − | − | − |
| 5.5 | + | − | − | − | − | − | − | − |
| 5.0 | − | − | − | − | − | − | − | − |

“+”growth;
“−”no growth

TABLE 6
Activity of PZA against M. tuberculosis H37Ra in
PZA-S1 medium containing L-Alanine as primary nitrogen
source under pH conditions from 5.0 to 8.5.

| | PZA (μg/ml) | | | | | | | |
|---|---|---|---|---|---|---|---|---|
| pH | 0 | 12.5 | 25 | 50 | 100 | 200 | 400 | 800 |
| 8.5 | + | + | + | + | − | − | − | − |
| 8.0 | + | + | + | + | + | − | − | − |
| 7.5 | + | + | + | + | + | − | − | − |
| 7.0 | + | + | + | + | + | − | − | − |
| 6.5 | + | + | + | + | + | − | − | − |
| 6.0 | + | + | + | + | − | − | − | − |
| 5.5 | + | + | − | − | − | − | − | − |
| 5.0 | − | − | − | − | − | − | − | − |

“+”growth;
“−”no growth

### Example 7. Endpoint Test Showing pH Changes in Growth Medium Following Growth of M. tuberculosis

pH is an important factor in the activity of PZA, such as the acidic pH conditions in the phagosome of a macrophage. An acidic pH also exists in TB lesions. However, the nature of this acidic pH environment is unclear. In general, it is believed that the inflammation triggered by mycobacterial infections may lead to the formation of an acidic environment. However, the mechanism of the acidic environment formation is unclear. Further, it has been reported that pH changes during M. tuberculosis growth, as reflected in endpoint test described herein, and that the direction to acidity or alkalinity was dependent on nutrient composition and culture time.

To detect pH changes in PZA-S1 media where PZA is active, pH was measured following growth of M. tuberculosis for 2-4 weeks in media containing L-Alanine, ammonium acetate, sodium lactate and sodium butyrate. The results in Table 7 show that the pH in PZA-S1-Plus media cultures containing L-Alanine, ammonium acetate, sodium lactate and sodium butyrate media became progressively more alkaline over time, and became progressively more acidic in PZA-S1-Plus media containing ammonium citrate dibasic, ammonium tartrate dibasic, ammonium chloride and ammonium nitrate between 2 to 4 weeks cultivation at 37° C. (Table 7). However, the pH was consistently neutral for 2 to 4 weeks in PZA-S1-Plus media containing ammonium formate, ammonium succinate or asparagine. In the L-Alanine medium culture, the pH increased from an initial pH of 6.8 to a pH of 8.5. By contrast, in the ammonium chloride medium culture, the pH decreased from an initial pH of 7.5 to a pH of 6. M. tuberculosis is usually walled off into fibrous capsules in the lung. These results suggest that the acidification of tuberculosis lesions appears may be due to M. tuberculosis growing in the enclosed phagosome space.

TABLE 7
Endpoint test showing pH changes in growth medium
following growth of M. tuberculosis H37Ra
for 2-4 weeks in different defined media.

| Media | Acidity | No change | Alkalinity |
|---|---|---|---|
| PZA-S1 | | | + |
| PZA-S1-Plus NA | | | + |
| SLac | | | + |
| SBA | | | + |
| PZA-S1-Minimal further supplemented | + | | |
| 0.25 g/L Ammonium citrate dibasic, | | | |
| 0.5 g/L citric acid, 10 ml/L glycerol | | | |
| PZA-S1-Minimal further supplemented | + | | |
| 0.25 g/L Ammonium tartrate dibasic, | | | |
| 0.5 g/L citric acid, 10 ml/L glycerol | | | |
| PZA-S1-Plus NC | + | | |
| PZA-S1-Minimal further supplemented | + | | |
| 0.25 g/L Ammonium nitrate, 0.5 g/L | | | |
| citric acid, 10 ml/L glycerol | | | |
| PZA-S1-Minimal further supplemented | | + | |
| 0.25 g/L Ammonium formate, 0.5 g/L | | | |
| citric acid, 10 ml/L glycerol | | | |
| PZA-S1-Minimal further supplemented | | + | |
| 0.25 g/L Ammonium succinate, 0.5 g/L | | | |
| citric acid, 10 ml/L glycerol | | | |
| PZA-S1-Minimal further supplemented | | + | |
| 2.0 g/L L-Asparagine, 0.5 g/L citric | | | |
| acid, 10 ml/L glycerol | | | |

A pH indicator, such as pH, may be used in a plate reader system or other automatic system to read the results. For example, phenol red is a pH indicator dye that exhibits a gradual transition from yellow to red over a pH range in cell cultures with pH values varying between pH 6.2 and pH 8.5. This color change is reflected in the change in wavelength peaks at 560 and 415 nm. For example, the absorption peak at 560 nm is obvious at pH 9.0, but almost disappears when the pH drops to 4.5. In contrast, as the culture becomes increasingly more acidic, an absorbance increase at 415 nm is obtained.

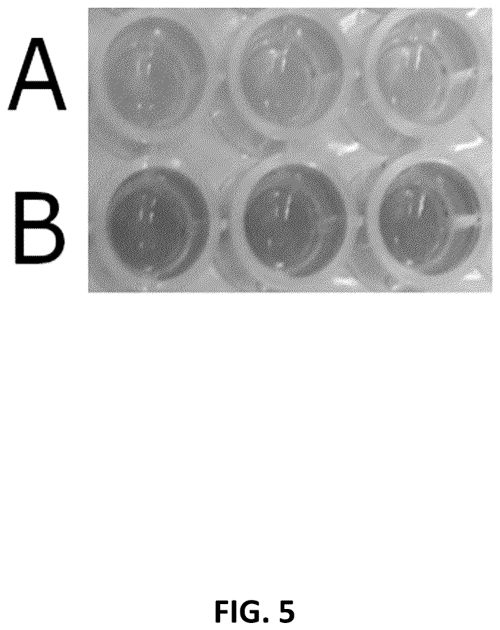

As shown in FIG. 5, a color change was observed when PZA susceptibility testing was carried out in PZA-S1 media. Table 8 shows the results of an experiment where phenol red was added at concentration of 15 μg/ml in different culture medias, including 7H9, L-Ala, SLac, and SBA. Following the growth of 200 μl M. tuberculosis cultures in 96 well plates, the OD₆₀₀, OD₄₁₅ and OD₅₆₀ readings were measured. The results of this experiment show increased signal amplification at OD₅₆₀ in M. tuberculosis cultures compared to control wells or A₅₆₀/A₄₁₅ in the PZA-S1 and PZA-S3 media containing phenol red. More particularly, a 9-fold increase in the absorbance at 560 nm was observed as the pH changed to alkaline from pH 6.8 in the L-Alanine defined media (PZA-S1) containing 15 μg/ml phenol red. No significant pH change was observed in the blank control (media only). To show changes in bacterial density, absorbance readings at 600 nm may be taken. In this Example, M. tuberculosis growth increased about 1.5-fold relative to the blank control. Accordingly, these parameters can be used markers for AST in an automatic plate reader system.

TABLE 8
Changes in optical density in several defined media.

| | Phenol red | 0 | | 15 | | 0 | | 15 | |
|---|---|---|---|---|---|---|---|---|---|
| | OD | 600 | G/C | 600 | G/C | 415 | G/C | 415 | G/C |
| 7H9 | Grow | 0.2244 | 3.63 | 0.2304 | 3.54 | 0.3841 | 3.52 | 0.7019 | 1.44 |
| | Control | 0.0618 | | 0.0651 | | 0.1090 | | 0.4859 | |
| PZA-S1 | Grow | 0.1119 | 2.31 | 0.1640 | 3.15 | 0.1842 | 2.31 | 0.3364 | 1.01 |
| | Control | 0.0485 | | 0.0521 | | 0.0796 | | 0.3328 | |
| SLac | Grow | 0.0453 | 0.93 | 0.0968 | 1.66 | 0.0586 | 0.90 | 0.1404 | 0.34 |
| | Control | 0.0486 | | 0.0584 | | 0.0654 | | 0.4173 | |
| SBA | Grow | 0.0496 | 0.95 | 0.0623 | 1.03 | 0.0637 | 0.82 | 0.3926 | 0.89 |
| | Control | 0.0521 | | 0.0603 | | 0.0774 | | 0.4428 | |

| | Phenol red | 0 | | 15 | | 0 | 15 |
|---|---|---|---|---|---|---|---|
| | OD | 560 | G/C | 560 | G/C | A560/A415 | A560/A415 |
| 7H9 | Grow | 0.2453 | 3.76 | 0.2805 | 3.08 | 0.64 | 0.40 |
| | Control | 0.0652 | | 0.0911 | | 0.60 | 0.19 |
| PZA-S1 | Grow | 0.1210 | 2.37 | 1.1318 | 8.99 | 0.66 | 3.36 |
| | Control | 0.0511 | | 0.1259 | | 0.64 | 0.38 |
| SLac | Grow | 0.0461 | 0.93 | 1.1806 | 4.07 | 0.79 | 8.41 |
| | Control | 0.0498 | | 0.2904 | | 0.76 | 0.70 |
| SBA | Grow | 0.0492 | 0.92 | 0.3801 | 1.59 | 0.77 | 0.97 |
| | Control | 0.0533 | | 0.2392 | | 0.69 | 0.54 |

The results of this study validate the usefulness of pH indicators for monitoring changes in M. tuberculosis growth.

### Example 8. First-Line Tuberculosis Drug Susceptibility in One Medium

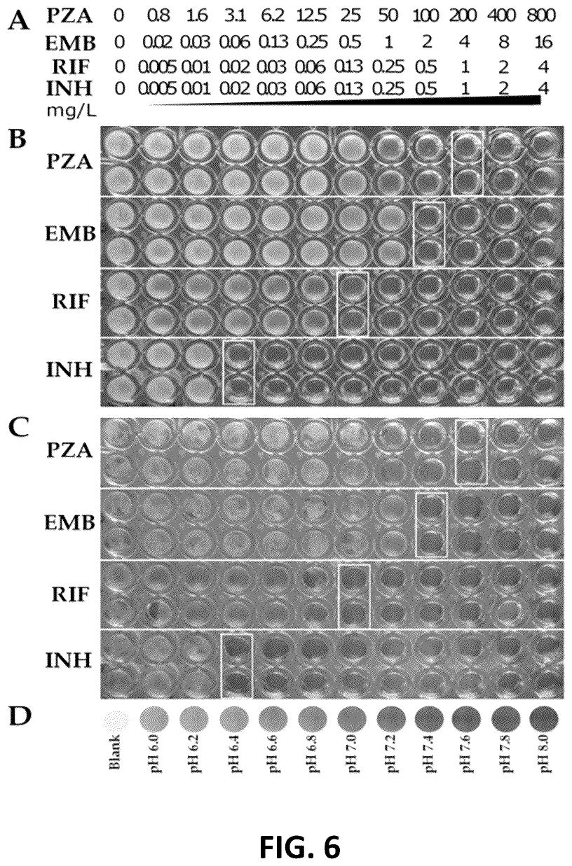

Current PZA susceptibility tests are performed at acidic pH in conventional liquid culture media, which is different from other tuberculosis drug susceptibility tests performed in neutral conditions. The present application provides a convenient, economical single culture system that can be employed for all tuberculosis drug susceptibility tests. To confirm this capability, the broth microdilution method was used to test for susceptibility of three other first-line tuberculosis drugs, rifampin (RIF), isoniazid (INH) and ethambutol (EMB) at neutral pH 6.8 using PZA-S1 media containing L-Alanine as a primary nitrogen source with glycerol and citric acid as primary carbon sources with or without phenol red. The results of this analysis are shown in FIG. 6 and further discussed below.

In the PZA-S1 media, the MICs for isoniazid, rifampin and ethambutol were experimentally determined as 0.06 μg/ml, 0.06 and 1.6 μg/ml, respectively. These results are consistent with the results obtained using the 7H9 medium at pH 6.8, which is routinely used. Additional TB drugs tested in the PZA-S1 medium, included linezolid (MIC, 2 μg/ml), bedaquiline (MIC, 0.064 μg/ml), moxifloxacin (MIC, 0.25 μg/ml), ethionamide (MIC, 0.5 μg/ml), moxifloxacin, streptomycin (MIC, 0.25 μg/ml). These experiments validate this approach for drug susceptibility testing of other TB drugs.

In another aspect, the defined media of the present application were supplemented with an antibiotic cocktail to prevent contamination of M. tuberculosis cultures. To test the effect of the antibiotic cocktail in those defined culture media, a drug susceptibility test was performed in the defined media supplemented with cycloheximide (10 μg/ml), carbenicillin (50 μg/ml), polymyxin B (25 μg/ml), trimethoprim (20 μg/ml). The results of this experiment showed that these PZA susceptibility tests were not affected by adding cycloheximide, carbenicillin, polymyxin B and trimethoprim.

### Example 9. Phenol Red-Based Microplate Reader for Antibiotic Susceptibility Testing by the Broth Microdilution Method

Phenol red is a pH indicator in culture medium that exhibits a gradual transition from yellow to pink over a pH range of 6.2 to 8.2. To evaluate the utility of using PZA-S1 media for determining the susceptibility to antibiotics, including first-line TB drugs, susceptibility tests of M. tuberculosis H37Ra were carried out using the broth microdilution method under neutral pH conditions. The concentrations of drugs in the 96-well plate were labeled as shown in FIG. 6, panel A shows the concentrations of first-line TB drugs in the 96-well plates shown in panels B and C. Each drug was progressively diluted at 2-fold serial dilutions and introduced to the wells in a 96 well plate (PZA from 0 to 800 μg/ml; EMB from 0 to 16 μg/ml; RIF and INH from 0 to 4 μg/ml).

FIG. 6, panel B shows TB drug susceptibility in M. tuberculosis grown in PZA-S1 medium (pH 6.8) in the presence of the TB drugs as indicated. Wells showing no growth are labeled with white rectangular boxes on the microplate for the first-line TB drug susceptibility tests. The results of this assay showed that the MICs of PZA, EMB, RIF and INH were 200 μg/ml, 2 μg/ml, 0.13 μg/ml and 0.02 μg/ml, respectively (FIG. 6, panel B). Each test on each drug sample was performed in duplicate.

In order to determine how the pH of M. tuberculosis changes during growth in the PZA-S1-Plus medium as set forth in FIG. 6, panel C, where phenol red was added at a concentration of 15 μg/ml in M. tuberculosis H37Ra cultures seeded on similar microtiter plates so that the pH of the cultures can be determined based on color. Wells showing no growth are labeled with white rectangular boxes on the microplate for the first-line TB drug susceptibility tests. Panel D shows color standards for pH 6.0 to 8.0 with phenol red in PZA-S1 medium and blank control.

Regardless of PZA or other first-line TB drugs in the culture, the pH at which the drugs completely inhibited the growth of M. tuberculosis in the wells or tube was ˜6.8 at 37° C. following 2 weeks of incubation (FIG. 6, panel C; see right side of white box). Further, based on the phenol red color standards depicted in FIG. 6, panel D, M. tuberculosis showed abundant growth at a pH of ˜7.0.

Additionally, the pH results of M. tuberculosis H37Ra cultures in PZA-S1 medium were measured at different time points, as shown in Table 9. These results show that the pH of M. tuberculosis cultures increased slightly from 6.8 to 7.2 at 37° C. over 18 days of incubation and then decreased to 6.5 over the total incubation time of 30 days in the PZA-S1 medium. In contrast, there was no significant change in the control group (Table 9). Therefore, PZA activity against M. tuberculosis occurred at a neutral pH at 37° C. throughout the cultivation period of the cells.

TABLE 9
Effect of pH as a function of M. tuberculosis incubation time.

| | Day of Incubation at 37° C. | | | | |
|---|---|---|---|---|---|
| | 0 | 9 | 15 | 18 | 30 |
| pH of culture | 6.8 | ~6.9 | ~7.0 | ~7.2 | ~6.5 |
| pH of control * | 6.8 | 6.8 | 6.8 | 6.8 | 6.8 |

### Example 10. Color Changes in Different Media Compositions Upon Growth of M. tuberculosis Grow at pH 6.8 for AST by the Broth Dilution Method

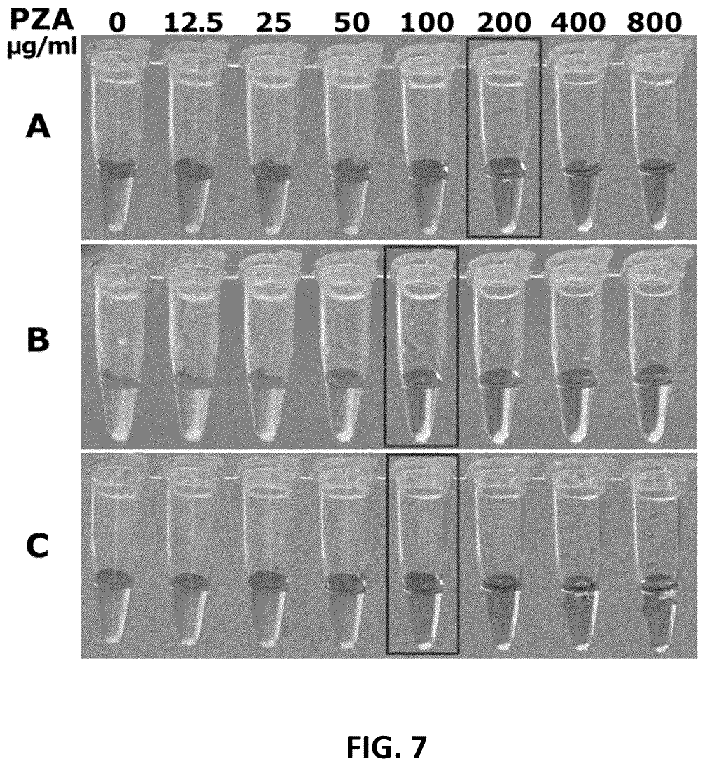

To further expand upon the study in Example 9, a further experiment was undertaken to investigate the extent to which changes in media can affect changes in pH. FIG. 7 shows the results of an analysis observing different color changes when M. tuberculosis grow at pH 6.8 in defined media using broth dilution method for 2 weeks incubation at 37° C.

Panel A shows a color change from light red to pink in media containing 2.0 g/L L-Ala, 0.25 g/L ammonium acetate, 0.2 g/L L-Leu, 0.2 g/L Met, 0.2 g/L L-Phe, 0.2 g/L L-Pro, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.0125% Tween 80 and 20 μg/ml phenol red.

Panel B shows a color change from light red to brown in media containing 2.0 g/L L-Ala, 0.2 g/L L-Leu, 0.2 g/L Met, 0.2 g/L L-Phe, 0.2 g/L L-Pro, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄, 0.0125% Tween 80, and 20 μg/ml phenol red.

Panel C shows no significant color change in media containing 2.0 g/L L-Ala, 0.02 g/L L-Gln, 0.08 g/L L-Leu, 0.08 g/L L-Phe, 0.08 g/L L-Ser, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/LS pyridoxine hydrochloride, 0.5 mg/Lo ZnSa₄, 0.0125% Tween 80 and 20 μg/ml phenol red.

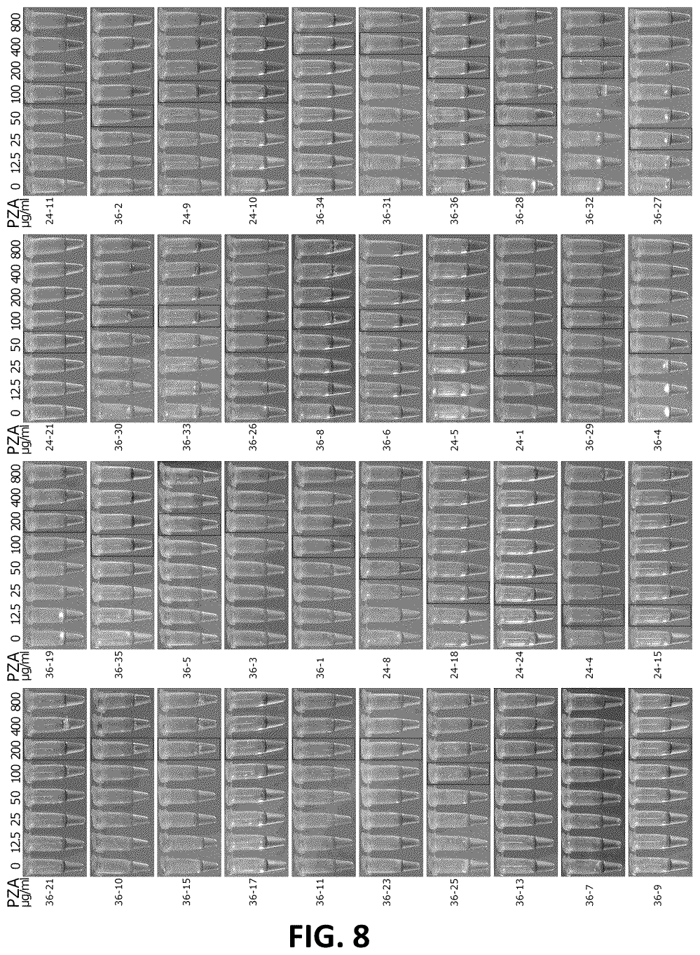

In another experiment, M. tuberculosis was inoculated in a variety of media differing in various combinations of amino acid(s), lipid(s) and carbon source(s) as indicated in Table 10 below. These cultures were incubated at neutral pH conditions at 37° C. in the presence of 20 μg/ml phenol red to facilitate determination of PZA susceptibility by the broth microdilution method. In addition, MICs were determined and changes in pH (indicated by color change) as a function of media composition were evaluated, as illustrated in FIG. 8. The results of the experiments depicted in FIG. 8 are summarized in Table 10.

TABLE 10
Determination of PZA susceptibility in different media inoculated with
M. tuberculosis and grown in neutral pH media at 37° C. for 20 days.

| | | MIC of PZA | | |
|---|---|---|---|---|
| | | (μg/ml) | | |
| | | 96 well | | |
| | Medium | plate | Tube | Color |
| 36-21 | PZA-S base + ADC 0.5 g/L + Ala 0.7 g/L + Ammonium | 800 | 200 | pink |
| | acetate 0.25 g/L + Gly 0.7 g/L + Leu 0.7 g/L + | | | |
| | Pyruvic acid sodium 0.25 g/L | | | |
| 36-10 | PZA-S base + ADC 0.5 g/L + Ala 0.7 g/L + Ammonium | 400 | 200 | pink |
| | acetate 0.25 g/L + Gly 0.7 g/L + Leu 0.9 g/L + Met | | | |
| | 0.2 g/L + Phe 0.2 g/L + Pro 0.2 g/L | | | |
| 36-15 | PZA-S base + ADC 0.5 g/L + Ala 0.7 g/L + Ammonium | 800 | 200 | pink |
| | acetate 0.25 g/L + Gly 0.7 g/L + Leu 0.7 g/L | | | |
| 36-17 | PZA-S base + ADC 0.5 g/L + Ala 1.0 g/L + Ammonium | >800 | 200 | pink |
| | acetate 0.25 g/L + 1.0 g/L Gly | | | |
| 36-11 | PZA-S base + ADC 0.5 g/L + Ala 1.0 g/L + Ammonium | 400 | 200 | pink |
| | acetate 0.25 g/L + 1.0 g/L Gly + Leu 0.2 g/L + Met | | | |
| | 0.2 g/L + 0.2 g/L Phe + 0.2 g/L Pro | | | |
| 36-23 | PZA-S base + ADC 0.5 g/L + Ala 1.0 g/L + Ammonium | >800 | 200 | pink |
| | acetate 0.25 g/L + Gly 1.0 g/L + Pyruvic acid | | | |
| | sodium 0.25 g/L | | | |
| 36-25 | PZA-S base + ADC 0.5 g/L + Ala 1.0 g/L + Gly 0.4 | 800 | 100 | — |
| | g/L + Glu 0.2 g/L + Met 0.4 g/L + Ser 0.4 g/L | | | |
| 36-13 | PZA-S base + ADC 0.5 g/L + Ala 2.0 g/L + Ammonium | >800 | 200 | pink |
| | acetate 0.25 g/L | | | |
| 36-7 | PZA-S base + ADC 0.5 g/L + Ala 2.0 g/L + Ammonium | 800 | 200 | pink |
| | acetate 0.25 g/L + Leu 0.2 g/L + Met 0.2 g/L + Phe | | | |
| | 0.2 g/L + Pro 0.2 g/L | | | |
| 36-9 | PZA-S base + ADC 0.5 g/L + Ala 2.0 g/L + Ammonium | 400 | 200 | pink |
| | acetate 0.25 g/L + Leu 0.2 g/L + Met 0.2 g/L + Phe | | | |
| | 0.2 g/L + Pro 0.2 g/L | | | |
| 36-19 | PZA-S base + ADC 0.5 g/L + Ala 2.0 g/L + Ammonium | >800 | 200 | pink |
| | acetate 0.25 g/L + Pyruvic acid sodium 0.25 g/L | | | |
| 36-35 | PZA-S base + ADC 0.5 g/L + Ala 2.0 g/L + Glu 0.5 g/L | 800 | 100 | — |
| 36-5 | PZA-S base + ADC 0.5 g/L + Ala 2.0 g/L + Gly 0.2 | 800 | 200 | pink |
| | g/L + Met 0.2 g/L + 0.25 g/L Pyruvic acid sodium + | | | |
| | Ser 0.2 g/L + Trp 0.2 g/L | | | |
| 36-3 | PZA-S base + ADC 0.5 g/L + Ala 2.0 g/L + Gly 0.2 | 800 | 200 | Light |
| | g/L + Met 0.2 g/L + Ser 0.2 g/L + Trp 0.2 g/L | | | yellow |
| 36-1 | PZA-S base + ADC 0.5 g/L + Ala 2.0 g/L + Leu 0.2 | 800 | 100 | Light |
| | g/L + Met 0.2 g/L + Phe 0.2 g/L + Pro 0.2 g/L | | | yellow |
| 24-8 | PZA-S base + Ala 0.7 g/L + Gln 0.02 g/L + Gly 0.7 | 200 | 50 | — |
| | g/L + His 0.2 g/L + Leu 0.7 g/L + Phe 0.08 g/L + | | | |
| | Ser 0.08 g/L | | | |
| 24-18 | PZA-S base + Ala 0.7 g/L + Gln 0.02 g/L + Gly 0.7 | 200 | 25 | — |
| | g/L + Leu 0.7 g/L | | | |
| 24-24 | PZA-S base + Ala 0.7 g/L + Gln 0.2 g/L + Leu 0.7 | | 25 | Light |
| | g/L + Ser 0.7 g/L | | | pink |
| 24-4 | PZA-S base + Ala 0.7 g/L + Gly 0.7 g/L + Glu 0.02 | 100 | ≤12.5 | — |
| | g/L + His 0.2 g/L + Leu 0.7 g/L + Phe 0.08 g/L + | | | |
| | Ser 0.08 g/L | | | |
| 24-15 | PZA-S base + Ala 0.7 g/L + Gly 0.7 g/L + Glu 0.02 | 400 | ≤12.5 | — |
| | g/L + Leu 0.7 g/L | | | |
| 24-21 | PZA-S base + Ala 0.7 g/L + Gly 0.7 g/L + Glu 0.2 | 400 | 50 | — |
| | g/L + Leu 0.7 g/L | | | |
| 36-30 | PZA-S base + Ala 1.0 g/L + Glu 1.0 g/L | 400 | 100 | pink |
| 36-33 | PZA-S base + Ala 1.0 g/L + Glu 2.0 g/L | 800 | 100 | pink |
| 36-26 | PZA-S base + Ala 1.0 g/L + Gly 0.4 g/L + Met 0.4 | 400 | 50 | Light |
| | g/L + Ser 0.4 g/L | | | yellow |
| 36-8 | PZA-S base + Ala 2.0 g/L + Ammonium acetate 0.25 | 400 | 100 | pink |
| | g/L + Leu 0.2 g/L + Met 0.2 g/L + Phe 0.2 g/L + | | | |
| | Pro 0.2 g/L | | | |
| 36-6 | PZA-S base + Ala 2.0 g/L + Butyric acid sodium 0.5 | 200 | 100 | — |
| | g/L + Gly 0.2 g/L + Met 0.2 g/L + Ser 0.2 g/L + Trp | | | |
| | 0.2 g/L | | | |
| 24-5 | PZA-S base + Ala 2.0 g/L + Gln 0.02 g/L + His 0.2 | 200 | 50 | — |
| | g/L + Leu 0.08 g/L + Phe 0.08 g/L + Ser 0.08 g/L | | | |
| 24-1 | PZA-S base + Ala 2.0 g/L + Glu 0.02 g/L + His 0.2 | 400 | 25 | — |
| | g/L + Leu 0.08 g/L + Phe 0.08 g/L + Ser 0.08 g/L | | | |
| 36-29 | PZA-S base + Ala 2.0 g/L + Glu 1.0 g/L | 400 | 100 | — |
| 36-4 | PZA-S base + Ala 2.0 g/L + Gly 0.2 g/L + Met 0.2 | 200 | 50 | — |
| | g/L + Ser 0.2 g/L + Trp 0.2 g/L | | | |
| 24-11 | PZA-S base + Ala 2.0 g/L + His 0.2 g/L + Leu 0.08 | 200 | 100 | — |
| | g/L + Phe 0.08 g/L + Ser 0.08 g/L | | | |
| 36-2 | PZA-S base + Ala 2.0 g/L + Leu 0.2 g/L + Met 0.2 | 200 | 50 | Light |
| | g/L + Phe 0.2 g/L + Pro 0.2 g/L | | | yellow |
| 24-9 | PZA-S base + Gln 0.02 g/L + His 0.2 g/L + Leu 0.08 | 400 | 100 | yellow |
| | g/L + Phe 0.08 g/L + Ser 0.08 g/L | | | |
| 24-10 | PZA-S base + Glu 0.02 g/L + His 0.2 g/L + Leu 0.08 | 400 | 100 | yellow |
| | g/L + Phe 0.08 g/L + Ser 0.08 g/L | | | |
| 36-34 | PZA-S base + Glu 1.0 g/L | >800 | 400 | pink |
| 36-31 | PZA-S base + Glu 2.0 g/L | >800 | 400 | pink |
| 36-36 | PZA-S base + Glu 2.0 g/L (without tween)# | >800 | 200-400 | — |
| 36-28 | PZA-S base + Gly 0.4 g/L + Glu 0.2 g/L + Met 0.4 | 400 | 50 | — |
| | g/L + Ser 0.4 g/L | | | |
| 36-32 | PZA-S base + Gly 0.4 g/L + Glu 2.0 g/L + Met 0.4 | >800 | 200 | pink |
| | g/L + Ser 0.4 g/L | | | |
| 36-27 | PZA-S base + Gly 0.4 g/L + Met 0.4 g/L + Ser 0.4 g/L | 200 | 25 | — |

*PZA-S base: 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, 0.5 mg/L ZnSO₄ and 0.0125% Tween 80 and 20 μg/ml phenol red. The pH was adjusted to 6.8 with 1M NaOH.

As shown in FIG. 8 and summarized in Table 10, following the growth of M. tuberculosis for 20 days, the cultures became more alkaline, where the color changed from light red to pink under certain media conditions (e.g., 36-21, 36-19), while in other media conditions, the cultures became more acidic, where the color changed from light red to yellow (e.g., 24-9, 24-10).

The foregoing results show the feasibility of using pH indicators in defined media to provide a visual basis for determining minimum inhibitory concentrations for antibiotics against M. tuberculosis. The results further illustrate that changes in media compositions and pH changes following growth in the same can be exploited to tailor the reaction conditions more effectively for ASTs and drug screening, especially for PZA-like drugs.

### Example 11. Broth Macrodilution Method for Determining MICs of Antibiotics in Neutral Conditions

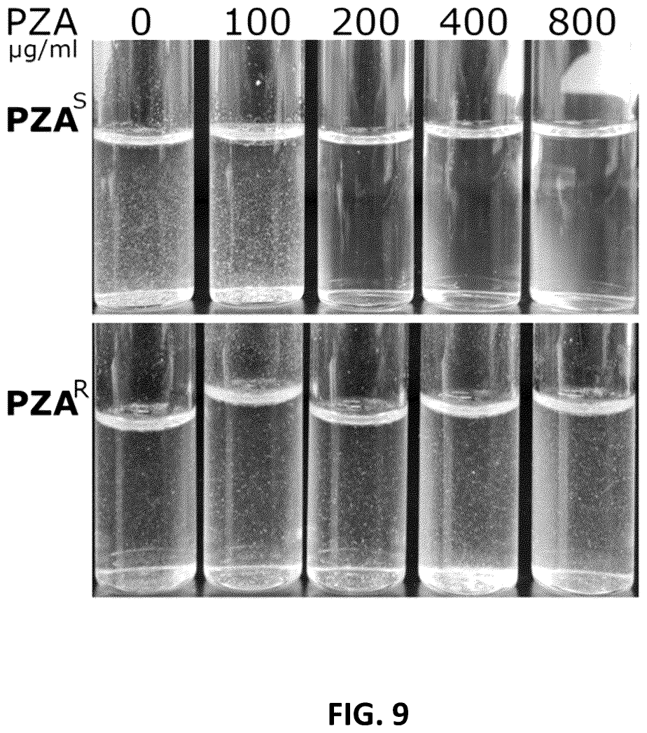

In another aspect, the broth macrodilution method was employed to determine MIC of antibiotics. Briefly, two fold serial dilutions of PZA from 800 μg/ml to 100 μg/ml (and no drug control) were prepared and added to wells containing M. tuberculosis H37Ra (PZA^{S}) and PZA-resistant strain (PZA^{R}) (#6 PncA mutation L159P) cultures at standard inoculum sizes of ˜1×10⁵ CFU/ml to ˜5×10⁵ CFU/ml in PZA-S1 medium at pH 6.8 containing 2.0 g/L L-Ala, 0.5 g/L KH₂PO₄, 0.5 g/L MgSO₄, 0.5 g/L citric acid, 10 ml/L glycerol, 30 mg/L ferric ammonium citrate, 0.5 mg/L biotin, 1 mg/L pyridoxine hydrochloride, and 0.5 mg/L ZnSO₄ (FIG. 9).

Susceptibility of PZA was tested by using macrodilution method in test tubes containing the above-described cultures. As shown in FIG. 8, the growth of the susceptible H37Ra (PZA^{S}) strain was inhibited at concentration of 200 μg/ml PZA, which is equivalent to the result obtained by the broth microdilution method. The resistant strain grew at a PZA concentration of 800 μg/ml PZA (FIG. 9). These results show that MICs of antibiotics can be determined at neutral pH in vitro by inhibiting M. tuberculosis visible growth by the broth macrodilution method utilizing the compositions and conditions described herein.

### Example 12. Agar Proportion Method for Determining MICs of Antibiotics in Neutral Conditions

For the agar proportion method, a drug susceptibility test was carried out according to the instructions of the Clinical and Laboratory Standards Institute. Serial dilutions were prepared. The PZA-containing and drug-free control agar plates were inoculated with 50 μl of a M. tuberculosis suspension. The drug-free control plates were inoculated with a 1:100 dilution of the M. tuberculosis suspension plated on the PZA-containing plates. PZA concentrations in the media were either 0, 25, 50, 100, 200, 400 or 800 μg/ml. The inoculated culture media were incubated at 37° C. for 4 weeks. Plates cultured in drug-free media having between 50 to 150 colonies per quadrant on the agar plate, were compared to the number of colonies in the corresponding PZA-containing plates. The proportion of PZA resistant M. tuberculosis in the plates was determined and expressed as a percentage of the total population of M. tuberculosis plated on the drug-containing plates. Where there are more colonies on the drug plate than the no drug control plate, the M. tuberculosis isolate is considered “resistant” (≥1%). The results of this assay are shown in Table 10.

TABLE 11
Percentage of CFU in PZA-agar vs. no PZA control plate.

Percentage of CFU to Z 0

| Strain | Z25 | Z50 | Z100 | Z300 |
|---|---|---|---|---|
| H37Ra | 1.20 | 0.14 | 0.02 | 0.08 |
| Z6 | 9.33 | 10.00 | 8.65 | 12.47 |
| Z98 | 6.76 | 6.62 | 5.15 | 5.81 |
| Z186 | 1.07 | 1.09 | 1.01 | 1.08 |
| ORA48 | 3.74 | 3.48 | 3.39 | 3.00 |
| ORA76 | 2.26 | 1.83 | 2.33 | 2.31 |
| ORA136 | 3.70 | 4.19 | 3.37 | 2.56 |

In Table 11, the percentage of CFU in the PZA-containing agar plates to the no PZA control plates was calculated. In this case, the MIC of PZA was defined as the concentration less than 1% CFU of the no PZA control plate. The MIC of PZA susceptible strain H37Ra was 50 μg/ml. In contrast, the MIC of PZA in the Z6, Z98, Z186 PZA-resistant strain with mutation in pncA gene, ORA48, ORA76, and the ORA 136 PZA-resistant strain with mutation in panD gene were greater than 300 μg/ml.

### Example 13. Next-Generation PZA Drug Screening

TB lesions are extremely complex, dynamic and respond differently to treatment. In addition, the metabolic regulation in M. tuberculosis is highly adaptive, thereby necessitating long-term treatment. Novel drugs against refractory M. tuberculosis populations are eagerly needed to shorten tuberculosis therapy. PZA plays role to shorten Tb treatment from 9-12 to 6 months. However, PZA is active at acidic pH in vitro in previous studies. Because of the instability of pH in the culture, it is not suitable to screen PZA-like drug under acidic conditions in vitro. Originally, PZA was discovered in an animal model directly. For large-scale drug screening, it is unfeasible to perform large-scale TB drug screening in animal models. The activity of PZA in neutral pH conditions with the PZA-S1-Plus media of the present application suggests the possibility for large-scale screening of PZA-like drugs in a high throughput format.

With the foregoing in mind, four sets of media were used in a microdilution screening assay to find broad spectrum PZA-like drugs for TB treatment. The four media include 7H9 medium, PZA-S1 medium, SLac medium with lactate as the primary carbon source, and SBA medium with butyric acid as the primary carbon source. In view of the characteristics of PZA activity in these media, the drug screen is expected to identify PZA-like drugs similarly sharing with PZA the previously observed property of PZA being inactive or barely active in 7H9 medium under neutral pH conditions. The latter accounts for the fact that conventional PZA susceptibility tests are carried out under acidic conditions.

The results of the drug screening assay in neutral pH conditions are summarized in Table 12. As shown in Table 12, a number of PZA-like drugs have been identified with greater activity (lower MICs) against M. tuberculosis in PZA-S1, SLac and SBA medium than PZA itself. In addition, the anti-TB activities of the compounds in Table 12 are significantly greater in the compositions of the present application than in 7H-9 media. Indeed, many of the compounds in Table 12 exhibit MIC values in 709 much higher than would be observed in conventional assays. Accordingly, the PZA-like compounds identified herein may further shorten TB treatment times alone or in combination with other TB drugs. Taken together, the data suggest that the compositions and screening methodology of the present application open a window into new TB compounds that would not have been otherwise uncovered using conventional TB drug screening assays known in the art.

TABLE 12
PZA-like compounds active against M. tuberculosis.

| | | | MIC (μg/ml) in the media | | | |
|---|---|---|---|---|---|---|
| NSC | Name | MW | 7H9 * | L-Ala | SLac | SBA |
| 13128 | Nicotinamide | 122.12 | 800 | 400 | 400 | 400 |
| 14911 | Pyrazinamide | 123.11 | 800 | 200 | 200 | 100 |
| 23516 | Risocaine | 179.22 | 800 | 0.12 | 0.25 | 0.25 |
| 41531 | Benzocaine (or NSC4688) | 165.19 | 800 | 0.26 | 0.26 | 0.26 |
| 128464 | Butamben | 193.24 | 800 | 0.06 | 0.06 | 0.06 |
| 401005 | Dtxsid30930780 | 354.4 | 141.76 | 35.44 | 8.86 | 2.22 |
| 84093 | 7-(Phenyl(4-toluidino)methyl)-8-quinolinol | 340.4 | 136.16 | 17.02 | 2.13 | 2.13 |
| 186200 | (5E)-5-Hydroxyimino-2,3,5- | 340.4 | 136.16 | 17.02 | 2.13 | 34.04 |
| | triphenylpentanenitrile | | | | | |
| 57654 | Hesperetin | 302.28 | 120.91 | 7.56 | 1.89 | 7.56 |
| 45114 | (Methyl(2,3,5-trichloro-6- | 298.5 | 119.40 | 29.85 | 3.73 | 14.93 |
| | hydroxybenzyl)amino)acetic acid | | | | | |
| 73753 | 5-(2,4-Dichlorophenoxy)-2-thioxo-1H- | 289.14 | 115.66 | 14.46 | 1.81 | 14.46 |
| | pyrimidin-4-one | | | | | |
| 55636 | Fenticlor | 287.2 | 114.88 | 1.80 | 0.92 | 0.92 |
| 123538 | Biochanin | 284.26 | 113.70 | 7.11 | 0.45 | 7.11 |
| 114344 | Ascochitine | 276.28 | 110.51 | 27.63 | 1.73 | 6.91 |
| 407292 | Dihydronaringenin* | 274.3 | 109.72 | 1.71 | 1.71 | 3.43 |
| 614911 | 3-Amino-N-(diaminomethylidene)-6-methyl- | 270.3 | 108.12 | 13.52 | 27.03 | 27.03 |
| | 5-phenylpyrazine-2-carboxamide | | | | | |
| 36586 | Genistein | 270.24 | 108.10 | 13.51 | 1.69 | 13.51 |
| 43318 | Pinocembrin | 254.24 | 101.70 | 6.36 | 0.41 | 6.36 |
| 56769 | Dioxybenzone | 244.24 | 97.70 | 24.42 | 0.76 | 24.42 |
| 7317 | Methyl 3,5-dinitrobenzoate | 226.1 | 90.44 | 11.31 | 5.65 | 5.65 |
| 122987 | Chembl4296951 | 221.29 | 88.52 | 1.38 | 5.53 | 11.06 |
| 406847 | Propyl 4-nitrobenzoate | 209.2 | 83.68 | 5.23 | 5.23 | 10.46 |
| 6763 | Ethyl 4-nitrobenzoate | 195.2 | 78.08 | 9.76 | 4.88 | 4.88 |
| 32740 | Ethyl 3-Nitrobenzoate | 195.2 | 78.08 | 2.44 | 2.44 | 4.88 |
| 46728 | Crassin acetate | 376.5 | 75.30 | 37.65 | 4.71 | 9.41 |
| 1327 | Methyl 3-Nitrobenzoate | 181.2 | 72.48 | 9.06 | 2.27 | 4.53 |
| 2902 | 4-Nitrobenzamide | 166.1 | 66.44 | 16.61 | 8.31 | 33.22 |
| 37327 | 3-Nitrobenzamide | 166.1 | 66.44 | 66.44 | 66.44 | 33.22 |
| 335306 | Closantel | 663.1 | 66.31 | 4.14 | 4.14 | 4.14 |
| 38270 | Olivomitsin | 1197.3 | 59.87 | 7.48 | 3.83 | 3.83 |
| 36962 | 3-Aminobenzamide | 136.2 | 54.48 | 13.62 | 13.62 | 13.62 |
| 36963 | 4-Aminobenzamide | 136.2 | 54.48 | 6.81 | 3.41 | 13.62 |
| 38768 | 2-Aminobenzamide | 136.2 | 54.48 | 54.48 | 54.48 | 54.48 |
| 747974 | RALOXIFENE, Raloxifene Hydrochloride | 473.6 | 47.36 | 2.96 | 2.96 | 2.96 |
| 320656 | 4-Methoxy-2-pyridin-2-yl-1H-benzimidazole | 225.3 | 45.06 | 5.63 | 1.41 | 11.27 |
| 31867 | Norlobaridone | 398.4 | 39.84 | 4.98 | 0.64 | 2.49 |
| 91340 | 1-(4-Chlorophenoxy)-3-[4-(3- | 381.3 | 38.13 | 4.77 | 4.77 | 9.53 |
| | chlorophenyl)piperazin-1-yl]propan-2-ol | | | | | |
| 36437 | Crassin acetate | 376.5 | 37.65 | 9.41 | 2.35 | 4.71 |
| 227186 | [(3R,5R,6S)-6-[8-Chloro-2-hydroxy-3-[[4- | 697.1 | 34.86 | 0.56 | 0.56 | 0.56 |
| | hydroxy-3-(3-methylbut-2- | | | | | |
| | enyl)benzoyl]amino]-4-oxochromen-7- | | | | | |
| | yl]oxy-5-hydroxy-3-methoxy-2,2- | | | | | |
| | dimethyloxan-4-yl] 5-methyl-1H-pyrrole-2- | | | | | |
| | carboxylate | | | | | |
| 123389 | Hydroberberubin | 325.4 | 32.54 | 16.27 | 8.14 | 16.27 |
| 38010 | Chaulmoogric acid, ethyl ester | 308.5 | 30.85 | 3.86 | 1.93 | 1.93 |
| 228150 | 4-(4-Methylphenyl)sulfinyl-7-nitro-2,1,3- | 303.3 | 30.33 | 7.58 | 3.79 | 3.79 |
| | benzoxadiazole | | | | | |
| 2952 | Dehydroabietic acid | 300.4 | 30.04 | 3.76 | 1.88 | 7.51 |
| 40306 | 7-Chloro-8-methyl-2-phenylquinoline-4- | 297.7 | 29.77 | 7.44 | 3.72 | 7.44 |
| | carboxylic acid | | | | | |
| 44175 | Polyporic acid | 292.3 | 29.23 | 7.31 | 3.65 | 3.65 |
| 7962 | 3-[4-(2-Methylbutan-2-yl)phenoxy]benzoic | 284.3 | 28.43 | 3.55 | 0.45 | 3.55 |
| | acid | | | | | |
| 774769 | Entrectinib; N-(5-(3,5-difluorobenzyl)-1H- | 560.6 | 28.03 | 7.01 | 3.50 | 3.50 |
| | indazol-3-yl)-4-(4-methyl-1-piperazinyl)-2- | | | | | |
| | (tetrahydro-2H-pyran-4-ylamino)benzamide | | | | | |
| 42135 | n4-(4-bromophenyl)pyrimidine-2,4,6- | 280.12 | 28.01 | 7.00 | 3.50 | 14.01 |
| | triamine | | | | | |
| 34875 | Naringenin | 272.25 | 27.23 | 6.81 | 1.70 | 6.81 |
| 327444 | 1-(1-Aminocyclohexyl)-5,6-dihydro-4H-2,1- | 268.4 | 26.84 | 6.71 | 3.36 | 13.42 |
| | benzothiazole-3-thiol | | | | | |
| 768068 | Cobimetinib | 531.3 | 26.57 | 13.28 | 3.32 | 3.32 |
| 40275 | 2-Naphthalenecarboxylic acid, 3- | 263.29 | 26.33 | 3.29 | 0.84 | 3.29 |
| | (phenylamino)- | | | | | |
| 37955 | 2-(Allyloxy)-3,5-dipropylbenzamide | 261.36 | 26.14 | 13.07 | 3.27 | 13.07 |
| 134058 | 5-Methylsulfanyl-6-phenylsulfanyl-2H-1,2,4- | 251.3 | 25.13 | 6.28 | 3.14 | 3.14 |
| | triazin-3-one | | | | | |
| 402600 | Chlorosalicylamide 0.5%, | 247.7 | 24.77 | 1.55 | 0.79 | 1.55 |
| | Chlorosalicylanilide, 5-Chlorosalicylanilide | | | | | |
| 311727 | 5-Nitro-2-pyrimidin-4-ylsulfanyl-1,3-thiazole | 240.3 | 24.03 | 0.77 | 0.77 | 0.77 |
| 51331 | 4-Phenylsulfanylquinazoline | 238.3 | 23.83 | 11.92 | 2.98 | 11.92 |
| 57103 | 5,7-Dinitroquinolin-8-ol | 235.2 | 23.52 | 0.75 | 0.75 | 0.75 |
| 11149 | 4-(2-Chlorobenzyl)benzene-1,3-diol | 234.68 | 23.47 | 5.87 | 1.47 | 2.93 |
| 11150 | 4-(4-Chlorobenzyl)benzene-1,3-diol | 234.68 | 23.47 | 5.87 | 1.47 | 2.93 |
| 126405 | (2,3,4,5-Tetrachlorocyclopenta-2,4-dien-1- | 231.9 | 23.19 | 5.80 | 5.80 | 5.80 |
| | ylidene)hydrazine | | | | | |
| 108235 | Phenol, 4-((2,6-diamino-3-pyridinyl)azo)- | 229.24 | 22.92 | 11.46 | 5.73 | 11.46 |
| 76627 | Ossamycin | 912.2 | 22.81 | 5.7 | 5.7 | 11.4 |
| 93817 | 2-Benzoxazolethiol, 5-phenyl- | 227.28 | 22.73 | 1.42 | 0.73 | 0.73 |
| 9358 | 4-(2-Pyridylazo)-N,N-dimethylaniline | 226.28 | 22.63 | 5.66 | 1.41 | 1.41 |
| 13051 | 4-[(6-Chloro-2-methoxyacridin-9-yl)amino]- | 406.9 | 20.35 | 2.54 | 1.30 | 2.54 |
| | 5-methyl-2-propan-2-ylphenol | | | | | |
| 150982 | 2-(2-Nitrovinyl)-1H-indole | 188.18 | 18.82 | 9.41 | 2.35 | 4.70 |
| 128068 | 8-Hydroxy-7-methylnaphthalene-2- | 183.21 | 18.32 | 9.16 | 2.29 | 9.16 |
| | carbonitrile | | | | | |
| 79486 | 3-(2,2-Diethoxyethylaminomethyl)-1,4- | 340.5 | 17.03 | 8.51 | 2.13 | 8.51 |
| | dimethyl-carbazole | | | | | |
| 785178 | Cassythicine | 325.36 | 16.27 | 8.13 | 4.07 | 4.07 |
| 303612 | Mequitazine | 322.5 | 16.13 | 8.06 | 4.03 | 8.06 |
| 35770 | Clomifene citrate | 598.1 | 14.95 | 1.91 | 0.96 | 1.91 |
| 147866 | Piperazine, 1-(3-amino-4-pyridyl)-4-(2,5- | 282.4 | 14.12 | 14.12 | 1.77 | 7.06 |
| | xylyl)- | | | | | |
| 785164 | Roemarine hydrochloride | 279.33 | 13.97 | 6.98 | 1.75 | 3.49 |
| 779217 | Osimertinib | 499.6 | 12.49 | 3.12 | 1.60 | 3.12 |
| 322661 | (6,8-Dichloro-2-(trifluoromethyl)-4- | 475.3 | 11.88 | 2.97 | 2.97 | 1.52 |
| | quinolinyl)(2-piperidinyl)methanol | | | | | |
| | methanesulfonate | | | | | |
| 281816 | Methiothepin maleate | 472.6 | 11.82 | 2.95 | 2.95 | 2.95 |
| 150954 | 4H-1,3-Thiazine, 5,6-dihydro-4,4-dimethyl- | 231.36 | 11.57 | 5.78 | 2.89 | 2.89 |
| | 2-(2-phenylethenyl)- | | | | | |
| 305798 | Enpiroline | 404.3 | 10.11 | 1.25 | 2.53 | 2.53 |
| 24113 | 3-(8-Chloro-7H-benzo[c]phenothiazin-7-yl)- | 368.9 | 9.22 | 2.31 | 1.18 | 2.31 |
| | N,N-dimethyl-1-propanamine | | | | | |
| 13573 | 8-Amino-6-methoxyquinoline | 174.2 | 8.71 | 8.71 | 17.42 | 8.71 |
| 85442 | Amiodarone hydrochloride | 681.8 | 8.52 | 4.26 | 1.09 | 2.18 |
| 9921 | Pentamidine | 340.4 | 8.51 | 2.13 | 1.09 | 4.26 |
| 300289 | Pinafide | 339.3 | 8.48 | 4.24 | 2.12 | 2.12 |
| 190374 | Ethoxazene (4-(4-ethoxy-phenylazo)- | 256.3 | 6.41 | 3.20 | 3.20 | 6.41 |
| | benzene-1,3-diamine) | | | | | |
| 317003 | 9H-Thioxanthen-9-one, monohydriodide | 456.3 | 5.70 | 2.85 | 1.46 | 2.85 |
| 3852 | 5-Nitroso-8-hydroxyquinoline | 174.2 | 4.36 | 2.18 | 1.09 | 2.18 |
| 343257 | Monensin sodium salt | 692.9 | 4.33 | 4.33 | 8.66 | 4.33 |
| 30263 | Methyl 4-Amino-2-Hydroxybenzoate | 167.2 | 4.18 | 1.05 | 1.05 | 4.18 |
| 3904 | Chloroxine | 214.04 | 4 | 0.03 | 0.03 | 0.03 |
| 338106 | Chembl4296947 | 240.22 | 3.00 | 1.50 | 0.77 | 1.50 |
| 30205 | N-(4-ethoxyphenyl)acridin-9-amine | 314.4 | 1.97 | 1.97 | 1.97 | 3.93 |
| 228155 | 7-Nitro-4-(1-oxidopyridin-1-ium-2- | 290.26 | 0.93 | 0.93 | 0.93 | 0.93 |
| | y1)sulfanyl-2,1,3-benzoxadiazole | | | | | |
| 373981 | Naphtho(2,1-b)furan-1-ethanol, 2-nitro- | 257.24 | 0.82 | 0.82 | 0.82 | 0.82 |
| 260594 | 4-[(1-Methyl-6-nitroquinolin-4- | 504.5 | 0.81 | 0.81 | 0.40 | 1.61 |
| | ylidene)amino]-N-[4-[(1-methylpyridin-4- | | | | | |
| | ylidene)amino]phenyl]benzamide | | | | | |
| 133100 | (2,15,17,27,29-Pentahydroxy-11-methoxy- | 697.8 | 0.56 | 0.56 | 0.56 | 0.56 |
| | 3,7,12,14,16,18,22-heptamethyl-6,23-dioxo- | | | | | |
| | 8,30-dioxa-24- | | | | | |
| | azatetracyclo[23.3.1.14,7.05,28]triaconta- | | | | | |
| | 1(29),2,4,9,19,21,25,27-octaen-13-yl) acetate | | | | | |
| 56410 | Porfiromycine | 348.35 | 0.28 | 0.28 | 0.56 | 0.28 |

* MIC ≥ the value in 7H9 media

The above results identify a number of prospective compounds as next-generation PZA drugs for improving the efficacy and shortening of TB treatments in much the same way as PZA combined with other TB drugs.

The above description is for the purpose of teaching a person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is in part reflected in the following claims. The claims may cover the claimed components and steps in any sequence which is effective to meet the objectives there intended, unless the context specifically indicates the contrary.

1. A composition for facilitating growth of M. tuberculosis, comprising:
(i) one or more nitrogen sources comprising one or more amino acids or salts thereof, alone or in combination with one or more ammonium salts;
(ii) one or more carbon sources comprising glycerol, butyric acid, lactate, cholesterol, pyruvic acid, dextrose, citric acid, or a combination thereof;
(iii) one or more salts comprising MgSO₄, KH₂PO₄, K₂HPO₄, NaH₂PO₄, or a combination thereof; and
(iv) one or more supplementary nutrients comprising ZnSO₄, pyridoxine hydrochloride, biotin, Tween 80, oleic acid, albumin, ferric ammonium citrate, catalase, or a combination the
wherein the composition is formulated to support antibiotic susceptibility testing of M. tuberculosis at a beginning pH between 6.2 to 8.0 at 35-37° C.

2. The composition of claim 1, wherein the one or more amino acids comprises a low concentration of asparagine, aspartic acid, glutamine, and/or glutamic acid.

3. The composition of claim 1, wherein the one or more amino acids comprise Ala, Arg, Lys, Ser, Thr, Ile, His, Pro, Val, Len, Met, Phe, Tyr, or a combination thereof.

4. The composition of claim 1, wherein the one or more nitrogen sources comprise one or more ammonium salts selected from the group consisting of ammonium acetate, ammonium succinate, ammonium chloride, ammonium citrate dibasic, ammonium tartrate dibasic, ammonium formate, ammonium nitrate, and combinations thereof.

5. The composition of claim 1, wherein the one or more carbon sources comprise glycerol, citric acid, cholesterol, butyric acid lactic acid, or a combination thereof.

6-12. (canceled)

13. The composition of claim 1, wherein the composition comprises albumin, dextrose, and catalase.

14. The composition of claim 1, wherein the composition comprises one or more antimicrobial agents.

15. The composition of claim 1, wherein the composition comprises a pH indicator.

16. (canceled)

17. A method for determining drug susceptibility of an antibiotic for treating a M. tuberculosis infection, comprising:
(a) inoculating a first medium comprising a composition accord of claim 1 with a clinical test sample of M. tuberculosis
(b) incubating the inoculated first medium in the presence or absence of the antibiotic under neutral pH conditions for a period of time and temperature sufficient to observe growth of the test sample in the absence of the antibiotic;
(c) comparing the growth of the test sample in the presence or absence of the antibiotic; and
(d) determining whether the test sample is sensitive or resistant to the antibiotic based on the comparison in step (c).

18. The method of claim 17, further comprising the steps of:
(a2) inoculating a second medium comprising the composition of claim 1 with a M. tuberculosis control sample that is sensitive to the antibiotic,
(b2) incubating the inoculated second medium in the presence or absence of the antibiotic under neutral pH conditions for a period of time and temperature sufficient to observe growth of control sample in the absence of the antibiotic;
(c2) comparing tile growth control sample with the growth of the test sample in media containing the antibiotic, and
(d2) determining whether the test sample is sensitive or resistant to the antibiotic based on the comparison in step (c2),
wherein a substantial growth inhibition of the control sample, and a substantial absence of growth inhibition of the test sample indicates that the test sample is resistant to the antibiotic, and
wherein substantial growth inhibition of both the control sample and, the test sample indicates that the test sample is sensitive to the antibiotic.

19. (canceled)

20. (canceled)

21. The method of claim 17, further comprising the step of determining the minimum inhibitory concentration of the antibiotic.

22. (canceled)

23. The Method of claim 17, wherein the antibiotic is selected from the group consisting of pyrazinamide, rifampin, rifapentine, isoniazid, ethambutol, bed aquiline, linezolid, moxifloxacin, delamanid, pretomanid, amikacin, clofazimine, cycloserine, rifabutin, levofloxacin, ethionamide, p-aminosalicylic acid; streptomycin and fluoroquinolone.

24. The method of claim 23, wherein the antibiotic is pyrazinamide, rifampin, isoniazid ethambutol.

25. The method of claim 24, wherein the antibiotic is pyrazinamide.

26. The method of claim 17, wherein each inoculated medium is incubated at a starting pH between 6.2 to 8.0.

27.-39. (canceled)

40. A method for screening a plurality of test compounds for their ability to inhibit the growth of Mycobacterium tuberculosis, comprising the steps of:
(a) inoculating a culture media comprising a composition of claim 1 with a M. tuberculosis isolate to from a control culture;
(b) inoculating in parallel a plurality of secondary cultures comprising the M. tuberculosis in step (a) to from a plurality of test cultures, wherein each of the plurality of test: cultures comprises a different test compound in the medium comprising the composition in step (a);
(c) incubating in parallel the first culture and the test cultures under neutral pH conditions for a period of time and temperature sufficient to observe growth of the control sample in the absence of the different test compounds in step (b);
(d) comparing ti e growth of the Mycobacterium tuberculosis isolate in the presence or absence of the test compound; and
(e) determining whether one or more of the test compounds inhibit growth of the M. tuberculosis isolate, based on the comparison in step (d).

41. The method of claim 40, wherein the comparison in step (d) comprises identifying one or more secondary cultures that are growth inhibited by a corresponding test compound.

42. The method of claim 40, wherein each culture medium comprises a pH indicator, and wherein the comparison in step (d) comprises detection of a change in the color of the culture medium following growth of a test culture.

43. The method of claim 42, wherein the change in color is detected by plate reader.

44. (canceled)

45. The method of claim 40, wherein one or more of the secondary cultures comprise an antibiotic that is known to inhibit growth of the M. tuberculosis isolate.

46.-52. (canceled)
